(19) United States
(12) Patent Application Publication
(10) Pub. No.: US 2023/0357724 A1
(43) Pub. Date: Nov. 9, 2023

(54) HUMAN UMBILICAL CORD MESENCHYMAL STEM CELL SHEETS AND METHODS FOR THEIR PRODUCTION

(71) Applicant: University of Utah Research Foundation, Salt Lake City, UT (US)

(72) Inventors: Teruo Okano, Salt Lake City, UT (US); Kyungsook Kim, Salt Lake City, UT (US); David Grainger, Salt Lake City, UT (US)

(73) Assignee: University of Utah Research Foundation, Salt Lake City, UT (US)

(21) Appl. No.: 17/422,677

(22) PCT Filed: Jan. 15, 2020
(86) PCT No.: PCT/US2020/013620

§ 371 Date: Jul. 13, 2021

Related U.S. Application Data
(60) Provisional application No. 62/793,199, filed on Jan. 16, 2019.

(51) Int. Cl. C12N 5/0775

(52) U.S. Cl. CPC C12N 5/0665; C12N 2500/98; C12N 2523/00; C12N 2539/10

(57) ABSTRACT

The disclosure provides a human umbilical cord mesenchymal stem cell sheet comprising one or more layers of confluent human umbilical cord mesenchymal stem cells (hUC-MSCs). The disclosure also provides method for producing hUC-MSC sheets comprising culturing hUC-MSCs in culture solution on a temperature-responsive polymer which has been coated onto a substrate surface of a cell culture support, wherein the temperature-responsive polymer has a lower critical solution temperature in water of 0-80° C.; adjusting the temperature of the culture solution to below the lower critical solution temperature, whereby the substrate surface is made hydrophilic and adhesion of the cell sheet to the surface is weakened; and detaching the cell sheet from the culture support.

### RELATED APPLICATIONS

This application claims priority to U.S. Provisional Pat. Application No. 62/793,199 filed on Jan. 16, 2019, the contents of which are incorporated herein in their entirety.

### BACKGROUND OF THE INVENTION

Mesenchymal stem cells (MSCs) are pluripotent somatic stem cells that can differentiate into osteoblasts, chondrocytes, nerve cells, skeletal muscle cells, vascular endothelial cells, and myocardial cells (Reyes et al., 2002, J. Clin. Invest. 109; 337-346; Toma et al., 2002, Circulation 105, 93-98; Wang et al., 2000, J. Thorac. Cardiovasc. Surg. 120, 999-1005; Jiang et al., 2002, Nature 41S, 41-49). Therapeutic properties of MSCs are proposed to derive from their intrinsic ability to 1) differentiate into diverse and distinct cell lineages, 2) produce an array of soluble bioactive factors central to cell maintenance, survival and proliferation, 3) modulate host immune responses, and 4) migrate as recruited to sites of injury to mitigate damage and promote healing (Squillaro et al., 2016, Cell Transplant, 25(5), 829-848). In particular, human umbilical cord MSCs (hUC-MSCs) are a promising cell source for stem cell therapy among diverse MSC types, with increasing clinical evidence (Bartolucci et al., 2017, Circ Res, 121(10), 1192-1204; Ichim et al., 2010, Int Arch Med, 3, 30; Riordan et al., 2018, J Transl Med, 16(1), 57; Tuma et al., 2016, Cell Transplant, 25(9), 1713-1721). Thus hUC-MSCs have great potential for a variety of therapeutic uses.

### SUMMARY OF THE INVENTION

In certain aspects, the disclosure relates to a human umbilical cord mesenchymal stem cell sheet comprising one or more layers of confluent human umbilical cord mesenchymal stem cells (hUC-MSCs). In certain embodiments, the cell sheet consists essentially of hUC-MSCs. In certain embodiments, at least 50% of cells in the cell sheet are hUC-MSCs. In certain embodiments, the cell sheet comprises an extracellular matrix. In certain embodiments, the extracellular matrix comprises one or more proteins selected from the group consisting of fibronectin, laminin and collagen. In certain embodiments, the cell sheet comprises cell adhesion proteins and cell-cell junction proteins. In certain embodiments, the cell junction proteins are selected from the group consisting of Vinculin, Integrin-β1, Connexin 43, β-catenin, Integrin-linked kinase and N-cadherin. In certain embodiments, the hUC-MSCs are isolated from the subepithelial layer of human umbilical cord tissue. In certain embodiments, the hUC-MSCs express a protein selected from CD44, CD73, CD105 and CD90. In certain embodiments, the hUC-MSCs express one or more cytokines selected from the group consisting of human growth factor (HGF), vascular endothelial growth factor (VEGF) and interleukin-10 (IL-10). In certain embodiments, expression of the one or more cytokines in the cell sheet is increased relative to a suspension of hUC-MSCs containing an equivalent number of cells. In certain embodiments, the cell sheet secretes tumor necrosis factor-α (TNF-α) into a culture solution at a rate of less than 50 pg/mL of culture solution/24 hours. In certain embodiments, the cell sheet expresses the one or more cytokines for at least 10 days after transplantation to a tissue in a host organism. In certain embodiments, the cell sheet expresses extracellular matrix proteins and cell junction proteins for at least 10 days after transplantation to a tissue in a host organism. In certain embodiments, the extracellular matrix proteins are selected from the group consisting of fibronectin, laminin and collagen. In certain embodiments, the cell junction proteins are selected from the group consisting of Vinculin, Integrin-β1, Connexin 43, β-catenin, Integrin-linked kinase and N-cadherin. In certain embodiments, initial seeded cell density of the hUC-MSCs in a cell culture support used to prepare the cell sheet is from 0.5 × 10⁴/cm² to 9 × 10⁵ /cm². In certain embodiments, the hUC-MSCs do not express one or more of CD31, CD45, Human Leukocyte Antigen - DR isotype (HLA-DR), Human Leukocyte Antigen - DP isotype (HLA-DP), or Human Leukocyte Antigen - DQ isotype (HLA-DQ). In certain embodiments, the hUC-MSCs comprise microvilli and filopodia. In certain embodiments, the cell sheet remains attached to a tissue in a host organism for at least 10 days after transplantation to the tissue.

In certain aspects, the disclosure relates to a composition comprising a cell sheet as described herein and a polymer-coated culture support that is removable from the cell sheet.

In certain aspects, the disclosure relates to a method for producing a human umbilical cord mesenchymal stem cell sheet comprising one or more layers of confluent human umbilical cord derived mesenchymal stem cells (hUC-MSCs), the method comprising: a) culturing hUC-MSCs in culture solution on a temperature-responsive polymer which has been coated onto a substrate surface of a cell culture support, wherein the temperature-responsive polymer has a lower critical solution temperature in water of 0-80° C.; b) adjusting the temperature of the culture solution to below the lower critical solution temperature, whereby the substrate surface is made hydrophilic and adhesion of the cell sheet to the surface is weakened; and c) detaching the cell sheet from the culture support.

In certain embodiments, the method comprises culturing the hUC-MSCs through multiple subcultures prior to the culturing step (a). In certain embodiments, 2 to 10 subcultures of the hUC-MSCs are performed prior to the culturing step (a). In certain embodiments, the culture solution is a xeno-free culture solution. In certain embodiments, the culture solution comprises human platelet lysate (hPL). In certain embodiments, the culture solution comprises fetal bovine serum (FBS). In certain embodiments, the culture solution comprises ascorbic acid. In certain embodiments, the adjusting step (b) is performed when the hUC-MSCs are confluent. In certain embodiments, the culturing step (a) comprises adding the hUC-MSCs to the culture solution at an initial cell seeding density from 0.5 × 10⁴/cm² to 9 × 10⁵ /cm². In certain embodiments, the hUC-MSCs are cultured in the culture solution on the temperature-responsive polymer for at least 24 hours before the adjusting step (b). In certain aspects, the disclosure relates to a cell sheet produced by the methods described herein.

In certain aspects, the disclosure relates to a method of transplanting a cell sheet to a subject comprising applying a cell sheet as described herein to a tissue of a subject. In certain embodiments of the methods and cell sheets described herein, the hUC-MSCs in the cell sheet are allogeneic to the subject. In certain embodiments of the methods and cell sheets described herein, the subject is a human.

### BRIEF DESCRIPTION OF THE DRAWINGS

FIG. 1 shows the cell sheet experimental protocol. Human umbilical cord stem cells (hUC-MSCs) were seeded on temperature responsive cell culture dishes (TRCD) and cultured to confluence in a 37° C. cell culture incubator. Cultured cells were detached from TRCDs as intact cell sheets within 1 hour at room temperature (RT), preserving their functional structures such as extracellular matrixes (ECMs) and cell-cell-junctions without using proteolytic enzyme treatments.

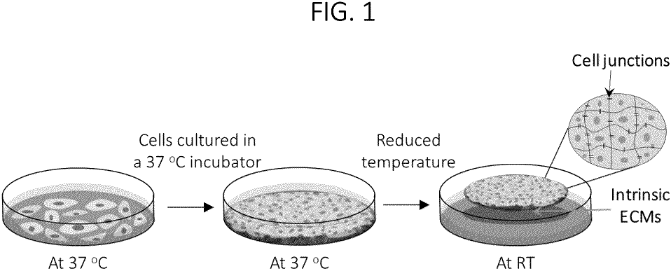

FIGS. 2A-2B shows hUC-MSC sheet morphological observations using cell passages 4, 6, 8, 10 and 12 seeded at 2 × 10⁴ cells/cm². (a) Morphology of passage 4, 6, 8, 10 and 12 cells observed using phase-contrast microscopy before sheet detachment. (b) successful fabrication of hUC-MSC sheets using passage 4, 6, 8 and 10 cells. In contrast, passage 12 cells detached as non-contiguous disconnected cellular structures. Scale bars = 100 µm.

FIGS. 3A-3C shows morphological observation, cell proliferation rate, and cell sheet fabrication for hUC-MSCs seeded at 2×10⁵, 1×10⁵ and 5×10⁴ initial cell numbers on a 35 mm diameter TRCD with a surface area of 9.6 cm². (a) cells cultured on TRCD and observed prior to sheet detachment. (b) Cell numbers counted using a hemacytometer after cells were seeded on TRCD until hUC-MSC sheets are observed. (c) cells detached as disconnected pieces at one day prior to confluence at 3, 4 and 5 days in 2×10⁵, 1×10⁵ and 5×10⁴ initial cell seeded groups, respectively. Intact cell sheets were successfully fabricated at 4, 5 and 6 day for seeding densities of 2×10⁵, 1×10⁵ and 5×10⁴ initial cell number groups, respectively. One day post-confluence, cultured cells spontaneously detach as aggregated forms without TRCD temperature changes at 5, 6 and 7 days for the 2×10⁵, 1×10⁵ and 5×10⁴ initial cell seeded groups, respectively. Scale bars indicate 100 µm in (a). Scale bars indicate 1 cm in (c).

FIGS. 4A-4D shows CD44 and CD90 positive expression in hUC-MSCs in cell suspension cultures (A and B) and in hUC-MSC sheets in vitro (C and D).

FIGS. 5A-5E shows cell-cell structural analysis using immunohistochemistry (IHC) and transmission electron microscopy (TEM). Cells successfully detached as sheets from TRCD by temperature changes at 4 days after seeding. Cell sheet were stained with ECM ((a) fibronectin and (b) laminin), and (c) cell junction β-catenin antibodies to confirm that cell sheets preserved their functional structures after detachment. In TEM images, the hUC-MSC sheet preserved their (d) ECMs and (e) cell-cell junction structures after detachment. Red arrow = ECMs; yellow arrow = cell junctions in the hUC-MSC sheet. Scale bars indicate 100 µm in (a-c). Scale bars (d) and (e) indicate 5 µm and 1 µm, respectively.

FIGS. 6A-6D shows cytokine analysis of human hepatocyte growth factor (HGF) and tumor necrosis factor-alpha (TNF-α) secreted from hUC-MSC sheets. hHGF (anti-inflammatory cytokine) and hTNF-α (pro-inflammatory cytokine) were detected in culture supernatant for cells cultured for 24 hours. (a) no significant differences in hHGF secretion in 2×10⁵, 1×10⁵, and 5×10⁴ initial cell seeded groups. (b); hTNF-α barely detected and not significantly different in 2×10⁵, 1×10⁵, and 5×10⁴ initial cell seeded groups. (c) Significant reductions in hHGF secreted from hUC-MSC sheets as passage increases. (d) hUC-MSC sheet fabricated using passage 4 cells secreted significantly lower amount of hTNF-α, compared to hUC-MSC sheet fabricated using passage 6, 8, 10, and 12 cells *p<0.05

FIGS. 7A-7E shows implanted hUC-MSC sheet retention in vivo. hUC-MSC sheets implanted within subcutaneous tissue in immuno-deficient mice. (c and d) At 10 days after implantation, hUC-MSC transplanted subcutaneous tissue sites were harvested for histological observation. In H&E-stained images, (b) the hu-MSC cell sheet was confirmed clearly in subcutaneous tissue implant sites compared to (a) normal subcutaneous tissue. Additionally, (e) abundant vascular structures are observed in cell sheet implanted groups. Arrows in (b) = implanted cell sheet; arrows in (e) = blood vessels. Scale bars (a and b) and (e) indicate 100 µm and 50 µm, respectively. Scale bars (c and d) indicated 0.5 cm.

FIGS. 8A-8B shows cell-cell junction related gene expression levels from hUC-MSC sheets. Gene expression levels of (a) integrin-linked protein kinase (ILK) and (b) N-cadherin (Ncad) associated with cell junctions in passage 12 were lower than that in passage 6.

FIGS. 9A-9C shows an illustration of the cell harvesting process. Human umbilical cord mesenchymal stem cells (hUC-MSC) were seeded on a 35 mm temperature responsive cell culture dish (TRCD) or tissue culture plate (TCP) and cultured for 5 days to reach confluence. hUC-MSC were harvested using 3 different methods which represents cell sheet technology, chemical disruption and physical disruption. A) cell sheet was harvested by temperature change, B) cells were treated with enzyme (trypsin) C) cells and harvested using a cell scraper.

FIGS. 10A-10D shows preparation of human umbilical cord mesenchymal stem cells (hUC-MSC) sheet (A) cells were cultured on conventional tissue culture plate (TCP) or temperature responsive cell culture dish (TRCD) for 5 days. Cell morphologies cultured on TCP and TRCD were observed using phase contrast microscope. (B) Cell number was counted using hemocytometer when they are cultured on TCP or TRCD for 100 hours. (C) The cells cultured on TRCD were detached as a sheet form by temperature reduction. (D) Histological analysis of the cell sheet was performed by H&E stain. Scale bars indicate 200 µm in A and D and 10 mm in C.

FIGS. 11A-11H shows morphological observation of hUC-MSC and hUC-MSC sheet. (A) morphology of cell surface was observed using scanning electron microscopy (SEM). (B) Microstructures of hUC-MSC sheets and hUC-MSCs were analyzed using transmission electron microscope (TEM). Dashed white arrows in (E) indicate ECMs, dark grey arrows in (H) indicate endoplasmic reticulum. Scale bar = 5 µm in SEM and TEM.

FIGS. 12A-12C shows cell dynamics-related protein expression analysis using western blot and immunohistochemistry. (A) western blot of F-actin, Vinculin and GAPDH in whole cell lysates (10 mg protein / lane). Immunostaining of (B) F-actin, (C) vinculin and DAPI (Blue). Scale bar = 10 µm.

FIGS. 13A-13C shows ECM protein expression analysis using western blot and immunohistochemistry. (A) western blot of fibronectin, laminin and GAPDH in whole cell lysates (10 mg protein / lane). Immunostaining of (B) fibronectin, (C) laminin and DAPI (Blue). Scale bar = 10 µm.

FIGS. 14A-14C shows cell-ECM and cell-cell junction proteins expression analysis using western blot and immunohistochemistry (A) western blot of integrin β-1, connexin 43 and GAPDH in whole cell lysates (10 mg protein / lane). Immunostaining of (B) integrin β-1, (C) connexin 43, and DAPI (Blue). Scale bar = 10 µm.

FIGS. 15A-15B shows live and dead cell assay (A) live and dead staining of cell sheet and cell suspension. Cells were stained by calcein and ethidium homodimer-1 immediately after cell detachment. Scale bar = 100 µm

FIG. 16 shows mechanosensor expression analysis using western blot. Western blot of Yes associated protein (YAP), phosphorylated-YAP and GAPDH in whole cell lysates (10 µg protein/ lane).

FIG. 17 shows hUC-MSC sheets prepared in culture medium containing human platelet lysate (hPL) (left) or fetal bovine serum (FBS) (right). The ruler shown is in cm.

FIGS. 18A-18B shows HGF expression in vivo in hUC-MSC sheets implanted within subcutaneous tissue of immuno-deficient mice. MSC sheet transplanted subcutaneous tissue sites were harvested for histological observation at 1 day (A) and 10 days (B) after implantation. The samples were stained with human HGF antibody for detection of HGF expression, and cell nuclei were stained with DAPI.

FIGS. 19A-19B shows hUC-MSC sheets produced with an initial cell density of 2×10⁴, 4×10⁴, 6×10⁴, 8×10⁴ or 10×10⁴ cells/cm² in the TRCD in cell culture media containing 20% FBS (A). Increasing initial cell density increased HGF gene expression in a dose-dependent manner (B).

FIGS. 20A-20B shows HLA DR, DP, DQ expression in hUC-MSC single cell suspension cultures (A) and cell sheets (B). HLA expression was measured from passage 4 to 12 in single cell suspension cultures (A). Percentages in (A) represent the percentage of cells expressing HLA. HLA-DR gene expression was not detectable in a hUC-MSC sheet, while cell sheets prepared from human adipose-derived stem cells (hADSC) or human bone marrow-derived mesenchymal stem cells (hBMSC) exhibited relatively high levels of HLA-DR gene expression (B).

FIGS. 21A - 21E shows hUC-MSC cultivation and doubling time influenced by FBS or hPL media. MSCs were cultured in FBS (A, C) or hPL media (B, D) at P6 (A, B) or P12 (C, D). Cell morphologies were observed (A-D) and cell numbers were counted during cell culture for all passage numbers to calculate cell doubling times (E).

FIGS. 22A -22D shows hUC-MSC differentiation potential influenced by FBS or hPL media. MSCs at P6 were cultured in FBS (A, C) or hPL (B, D) media before being induced for differentiation. Osteogenic differentiation (A, B) marked by Alizarin Red staining and adipogenic (C, D) differentiation marked by Oil Red O staining.

FIGS. 23A- 23L shows hUC-MSC phenotypes influenced by FBS or hPL media. P6 hUC-MSCs displayed positive expression of CD73, CD105, and CD90 and negative expression of MHC II, CD45, and CD31 in FBS (A-F) and hPL (G-L) media culture.

FIGS. 24A- 24O shows hUC-MSC sheet comparisons in FBS and hPL media. When cells reach confluence (A, C, E, and G) on TRCDs, MSC sheets were detached by culture temperature changes from 37° C. to RT. Cells cultured to over-confluence (B, D, F, and H) produced cell sheets in FBS media that successfully detached in sheet forms (B and F), but cell sheets prepared in hPL media detach from TRCDs spontaneously without any temperature changes at 37° C. (D and H). Cell sheets prepared in hPL media detached in sheet form faster than those in FBS media after temperature reduction to RT (I). H&E staining is preformed to observe MSC sheet structural changes in FBS (J) and hPL (K) media. Cytoskeletal structures of MSCs cultured in FBS (M) or hPL (N) media were imaged by phalloidin staining. Gene expression levels of β-actin and ITGB1 in FBS or hPL groups were assessed by q-PCR analysis (L, O). Scale bars represent 100 µm (A-D, J, and K), 1 cm (E-H), and 50 µm (M and N). *p<0.05 (N number = 3 -4).

FIGS. 25A-25K shows cell sheet-specific structure and property analysis using IHC and q-PCR. Fibronectin (Fb) (B, F), β-catenin (β-CTNN) (C, G), and MHC II (D, H) positive areas in MSC sheets prepared with FBS (A-D) or hPL (E-H) media were imaged and compared with negative control groups (A, E). Gene expression levels for Fb (I), β-CTNN (J), MHC II (K) in FBS and hPL groups were assessed. No significant (NS) differences in gene expression levels were observed for Fb (I), β-CTNN (J), and MHC II (K) (N number = 3 - 4). Scale bars represent 200 µm.

FIGS. 26A-26C shows cytokine secretion of human HGF from hUC-MSC sheets for 24 h (A). Cell numbers were counted immediately after supernatant was collected (B). Amounts of hHGF were normalized to cell number in each group (C).

### DETAILED DESCRIPTION OF THE INVENTION

This disclosure describes preparation and properties for human umbilical cord mesenchymal stem cell (hUC-MSC) sheets for improving MSC engraftment efficiencies and retention at target tissue sites in MSC therapies. Currently, injected MSC cell suspensions are harvested using enzymes that compromise MSC functions and engraftment capabilities, resulting in low tissue retention and survival, and sub-optimal therapeutic properties. Cell sheets created without enzymes and as living sheets with an extracellular matrix (ECM) and cell receptors intact, such as those described herein, can be physically placed on tissue sites with highly improved retention and engraftment efficiencies.

Human umbilical cord mesenchymal stem cells (hUC-MSCs) were used to prepare cell sheets in vitro in temperature-responsive cell culture dishes (TRCDs) coated with a temperature-responsive polymer. Confluent cell sheets formed at 4-6 days after seeding and were detached from the TRCD by cooling the cultures to room temperature. Various culture conditions were identified that allow for successful production of robust, uniform hUC-MSC sheets containing a monolayer of aggregated, confluent cells. These culture conditions included optimization of subculture (passage) number before adding cells to the TRCD, initial cell density in the TRCD, addition of fetal bovine serum (FBS) or cell growth factors such as human platelet lysate (hPL) to the cell culture solution, and culture time in the TRCD before detachment from the temperature-responsive polymer. The hUC-MSC sheets produced by these methods displayed several beneficial properties over current injected cell suspensions for improving allogeneic MSC cell therapy. These beneficial properties include sustained secretion of cytokines, a low HLA expression profile, intact hUC-MSC sheet retention on implant target tissue sites in vivo for 10 days, and new blood vessel recruitment into sheets on the target tissue. In addition, the harvested mono-layer hUC-MSC sheet retained tissue-like structures, extracellular matrices (ECMs), cell-cell junctions and cell-ECM junctions, and had higher cell survival rates compared to conventional chemical disruption methods such as trypsin treatment. With reliable topical tissue site placement, high engraftment efficiency, and long-term retention and survival in vivo, the hUC-MSC sheets produced by the methods described herein have the potential to greatly improve the therapeutic value of allogeneic cell therapy over the injected mesenchymal stem cell suspensions currently in use.

In some embodiments, other mesenchymal stem cells (MSCs) may be used to prepare a cell sheet with one or more of the beneficial characteristics of the hUC-MSC sheets described herein, including but not limited to sustained secretion of cytokines, a low HLA expression profile, intact MSC sheet retention on implant target tissue sites in vivo for 10 days, and new blood vessel recruitment into sheets on the target tissue.

### I. Human Umbilical Cord MSCs (hUC-MSCs)

The term “human umbilical cord mesenchymal stem cell” or “hUC-MSC” as used herein refers to a mesenchymal stem cell that has been isolated from a human umbilical cord.

Mesenchymal stem cells (MSCs) have a remarkable clinical potential to treat a wide range of debilitating diseases, mainly due to their unique immunomodulatory role and regenerative capacity (Caplan and Sorrell, 2015, Immunol Lett 168(2): 136-139). A convenient source for human MSCs is the umbilical cord, which is discarded after birth and provides an easily accessible and non-controversial source of stem cells for therapy (El Omar et al., 2014, Tissue Eng Part B Rev 20(5): 523-544). hUC-MSCs have been validated for safety and efficacy in human clinical trials as suspensions (Bartolucci et al., 2017, Circ Res, 121(10), 1192-1204). Moreover, hUC-MSCs have been successfully used in experimental animal disease models (Zhang et al., 2017, Cytotherapy 19(2): 194-199).

Methods for isolating MSCs from umbilical cords are known in the art and are described, for example, in U.S. Pat. No. 9,903,176, which is incorporated by reference herein in its entirety. The human umbilical cord comprises the umbilical artery, the umbilical veins, Wharton’s Jelly, and the subepithelial layer. In some embodiments, the hUC-MSCs are isolated from the subepithelial layer of the human umbilical cord. In some embodiments, the hUC-MSCs are isolated from Wharton’s Jelly of the human umbilical cord. Various cellular markers may be used to identify hUC-MSCs isolated from the subepithelial layer. For example, in some embodiments, the hUC-MSCs isolated from the subepithelial layer express one or more cell markers selected from CD29, CD73, CD90, CD146, CD166, SSEA4, CD9, CD44, CD146, and CD105. In a particular embodiment, the hUC-MSCs express CD73. In some embodiments, the hUC-MSCs isolated from the subepithelial layer do not express one or more cell markers selected from CD45, CD34, CD14, CD79, CD106, CD86, CD80, CD19, CD117, Stro-1, HLA-DR, HLA-DP and HLA-DQ. In a particular embodiment, the hUC-MSCs do not express HLA-DR, HLA-DP or HLA-DQ. In some embodiments, the cell sheets described herein are prepared with mesenchymal stem cells (MSCs) with low HLA expression, e.g. less than 5%, 4%, 3%, 2%, 1%, 0.5%, 0.4%, 0.3%, 0.2%, or 0.1% of the MSCs in the cell sheet express HLA (e.g. HLA-DR, HLA-DP and/or HLA-DQ).

hUC-MSCs in the umbilical cord are surrounded by extracellular matrix (ECM) and connected with other types of umbilical cord cells (e.g. endothelial cells, epithelial cells, muscle cells, and fibroblasts) through cell-cell junction structures. In contrast to endogenous hUC-MSCs in the umbilical cord, the hUC-MSC sheets described herein comprise a monolayer of aggregated confluent hUC-MSCs in which the hUC-MSCs are connected to other hUC-MSCs, not to other types of umbilical cord cells. The hUC-MSC sheets described herein also differ from harvested MSC suspensions in several ways. Suspensions of hUC-MSCs contain single cells that do not have an ECM or cell-cell junctions because the adhesive proteins in these cell-cell junctions must be removed (e.g. by trypsin treatment) to harvest cells from culture surfaces for preparation of the cell suspension culture. In contrast to singe cell suspensions of hUC-MSCs, the hUC-MSC sheets described herein contain both an ECM and cell-cell junctions among the hUC-MSCs that are generated during formation of the cell sheet. The intact ECM and cell-cell junctions facilitate adhesion of the hUC-MSC sheet to target tissue during transplantation to a host organism.

### II. Cell Sheets Produced From Human Umbilical Cord MSCs (hUC-MSCs)

In certain aspects the present disclosure relates to a human umbilical cord mesenchymal stem cell sheet comprising one or more layers of confluent human umbilical cord mesenchymal stem cells (hUC-MSCs). The term “human umbilical cord mesenchymal stem cell sheet” or “hUC-MSC sheet” as used herein refers to a cell sheet obtained by growing human umbilical cord mesenchymal stem cells on a cell culture support in vitro. The hUC-MSC sheets described herein are harvested as a sheet of one or more layers with a temperature shift using a temperature-responsive culture dish (TRCD) without any enzyme treatment. The hUC-MSC sheets maintain their sheet and shape by retaining tissue-like structures, actin filaments, extracellular matrix, intercellular proteins, and high cell viability, all of which are related to improved cell survival and cellular functions relevant to cell therapy. Accordingly, the cell sheets described herein may comprise structural features that improve cell survival and cell function, including an extracellular matrix, cell adhesion proteins and cell junction proteins. Thus, the hUC-MSC sheets prepared by the methods described herein have several beneficial characteristics compared to MSCs produced by other methods. For example, chemical disruption (proteolytic enzyme treatment) is widely used in cells harvested for stem cell therapy. However, the chemical disruption method is unable to maintain tissue-like structures of cells as well as cell-cell communication, since enzyme treatment disrupts the extracellular and intracellular proteins (cell-cell and cell-ECM junctions). Accordingly, protein cleavage by enzymes reduces cell viability and cellular functions relevant to cell therapy.

In some embodiments, the extracellular matrix comprises one or more proteins selected from the group consisting of fibronectin, laminin and collagen. In some embodiments, the cell junction proteins are selected from the group consisting of Vinculin, Integrin-β1, Connexin 43, β-catenin, Integrin-linked kinase and N-cadherin.

The hUC-MSCs in the cell sheets may also maintain additional structural features, such as microvilli and filopodia. Microvilli are cell membrane protrusions involved in a wide variety of cell functions, including absorption, secretion, and cellular adhesion. Filopodia are cytoplasmic projections that play a role in cell-cell interactions. Thus maintenance of these structural features may also help to maintain cell function and signaling.

In some embodiments, the cell sheet consists of hUC-MSCs. In some embodiments, the cell sheet consists essentially of hUC-MSCs. In some embodiments, at least 50%, 60%, 70%, 80%, 90%, 95%, 96%, 97%, 98% or 99% of cells in the cell sheet are hUC-MSCs. In some embodiments, 100% of the cells in the cell sheet are hUC-MSCs.

The hUC-MSCs may be added to the culture solution on the temperature-responsive polymer in the cell culture support at various cell densities to optimize formation of the cell sheet or its characteristics. For example, cytokine expression levels in the hUC-MSC may be optimized by controlling the initial cell density of the hUC-MSCs in the cell culture support (e.g. TRCD). In some embodiments, increasing the initial cell density of the hUC-MSCs in the cell culture support increases cytokine expression (e.g. HGF). In some embodiments, decreasing the initial cell density of the hUC-MSCs in the cell culture support decreases cytokine expression. In some embodiments the initial cell density of the hUC-MSCs in the cell culture support used for preparation of the cell sheet is from 0.5 × 10⁴/cm² to 9 × 10⁵/cm² In some embodiments, the initial cell density of the hUC-MSCs in the cell culture support is at least 0.5×10⁴, 1×10⁴, 2×10⁴, 3×10⁴, 4×10⁴, 5×10^{4,} 6×10⁴, 7×10⁴, 8×10⁴, 9×10⁴, 1×10⁵, 2×10⁵, 3×10⁵, 4×10⁵, 5×10^{5,} 6×10⁵, 7×10⁵, 8×10⁵, or 9×10⁵ cells/cm². Any of these values may be used to define a range for the initial cell density of the hUC-MSCs in the cell culture support. For example, in some embodiments, the initial cell density in the cell culture support is from 2×10⁴ to 1×10⁵ cells/cm², 4×10⁴ to 1×10⁵ cells/cm², or 1×10⁴ to 5×10⁴ cells/cm².

The hUC-MSC sheets described herein may be transplanted to a target tissue in a host organism (e.g. a human) for therapeutic uses. Transplantation of the hUC-MSC sheets to the target tissue may result in the formation of capillaries (angiogenesis) in the host tissue, as well as blood vessel formation between the transplanted cell sheet and the host tissue. This neocapillary formation is an important capability for sheet engraftment, cell viability and tissue regeneration. In addition, this new blood vessel recruitment into sheets on the target tissue suggests that implanted hUC-MSC sheets continually secrete paracrine factors to modulate engraftment.

In some embodiments, the hUC-MSC sheets express one or more cytokines, for example, one or more anti-inflammatory cytokines or one or more inflammatory cytokines. In some embodiments the anti-inflammatory cytokine is selected from human growth factor (HGF), fibroblast growth factor (FGF), vascular endothelial growth factor (VEGF) and interleukin-10 (IL-10). In some embodiments, the inflammatory cytokine is tumor necrosis factor-α (TNF-α). In some embodiments, expression of the cytokine (e.g. an anti-inflammatory cytokine or an inflammatory cytokine) in the cell sheet is increased relative to a suspension of hUC-MSCs containing an equivalent number of cells. In some embodiments, expression of the cytokine (e.g. an anti-inflammatory cytokine or an inflammatory cytokine) is decreased relative to a suspension of hUC-MSCs containing an equivalent number of cells. For some therapeutic uses, reducing secretion of inflammatory cytokines by the cell sheet would be beneficial. For example, in a particular embodiment, the cell sheet secretes tumor necrosis factor-α (TNF-α) into a culture solution in vitro at a rate of less than 100, 90, 80, 70, 60, 50, 40 or 30 pg/mL of culture solution/24 hours.

The hUC-MSC sheets described herein may continue to express cytokines after transplantation to a target tissue in a host organism. In some embodiments, the cell sheet expresses the cytokine for at least 1, 2, 3, 4, 5, 6, 7, 8, 9, 10, 15, 20, 25 or 30 days after transplantation to a tissue in a host organism. In some embodiments, the cell sheet expresses the cytokine for at least 1, 2, 3, 4, 5 or 6 months after transplantation to a tissue in a host organism.

The hUC-MSC sheets described herein may also continue to express extracellular matrix proteins and cell junction proteins after transplantation to a target tissue in a host organism. For example, in some embodiments the cell sheet expresses extracellular matrix proteins and/or cell junction proteins for at least 1, 2, 3, 4, 5, 6, 7, 8, 9, 10, 15, 20, 25 or 30 days after transplantation to a tissue in a host organism. In some embodiments, the cell sheet expresses the extracellular matrix proteins and/or cell junction proteins for at least 1, 2, 3, 4, 5 or 6 months after transplantation to a tissue of a host organism. In some embodiments the extracellular matrix proteins expressed in the cell sheet after transplantation are selected from fibronectin, laminin and collagen. In some embodiments the cell junction proteins expressed in the cell sheet after transplantation are selected from Vinculin, Integrin-β1, Connexin 43, β-catenin, Integrin-linked kinase and N-cadherin.

Current stem cell therapies often use cultured stem cells isolated from biopsies as injectable cell suspensions (Bayoussef et al., 2012, J Tissue Eng Regen Med, 6(10)). Injected cell suspensions typically exhibit lower engraftment into and retention within diseased organs or tissues (Devine et al., 2003, Blood, 101(8), 2999-3001). Loss of intact ECM and cell-cell junctions (i.e., communication) in stem cell suspensions through enzymatic disruption at harvest compromises stem cell function, engraftment and survival in vivo, and can limit therapeutic efficacy in vivo. In contrast, the methods of preparing hUC-MSC sheets described herein preserve intrinsic cell functional structures, improving attachment of the cell sheet to the target tissue after transplantation. For example, in some embodiments, the cell sheet remains attached to the target tissue in the host organism for at least 1, 2, 3, 4, 5, 6, 7, 8, 9, 10, 15, 20, 25 or 30 days after transplantation to a tissue in a host organism. In some embodiments, the cell sheet remains attached to the target tissue in the host organism for at least 1, 2, 3, 4, 5 or 6 months after transplantation to a tissue of a host organism.

Human leukocyte antigens (HLAs) are cell-surface proteins that make up the major histocompatibility complex (MHC) proteins in humans and are responsible for regulation of the immune system. HLAs corresponding to MHC class II (DP, DM, DO, DQ, and DR) present antigens from outside of the cell to T-lymphocytes. These antigens stimulate the multiplication of T-helper cells (CD4⁺ T cells), which in turn stimulate antibody-producing B-cells to produce antibodies to that specific antigen. Thus minimizing expression of HLAs would be beneficial in minimizing an immune response to hUC-MSC sheets transplanted into a host organism. In some embodiments, the hUC-MSC sheets described herein do not express one or more of Human Leukocyte Antigen - DR isotype (HLA-DR), Human Leukocyte Antigen - DP isotype (HLA-DP), or Human Leukocyte Antigen - DQ isotype (HLA-DQ). In some embodiments, less than 5%, 4%, 3%, 2%, 1%, 0.5%, 0.4%, 0.3%, 0.2%, or 0.1% of the hUC-MSCs in the cell sheet express HLA (e.g. HLA-DR, HLA-DP and/or HLA-DQ).

### III. Methods for Producing Human Umbilical Cord MSC (hUC-MSC) Sheets in Vitro

In certain aspects, the present disclosure relates to a method for producing a cell sheet comprising a monolayer of aggregated confluent human umbilical cord mesenchymal stem cells (hUC-MSCs), the method comprising:
a) culturing hUC-MSCs in culture solution on a temperature-responsive polymer which has been coated onto a substrate surface of a cell culture support, wherein the temperature-responsive polymer has a lower critical solution temperature in water of 0-80° C.;
b) adjusting the temperature of the culture solution to below the polymer lower critical solution temperature, whereby the substrate surface is made hydrophilic and adhesion of the cell sheet to the surface is weakened (e.g. by water penetration); and
c) detaching the cell sheet from the culture support.

Methods for isolating human umbilical cord mesenchymal stem cells (hUC-MSCs) are known in the art and are described, for example, in U.S. Pat. No. 9,803,176, which is incorporated by reference herein in its entirety. For example, hUC-MSCs may be isolated from the subepithelial layer of an umbilical by washing the umbilical cord to remove blood, Wharton’s Jelly, and any other material, and dissecting the subepithelial layer (SL) from the umbilical cord. The cord tissue may be washed multiple times in a solution of Phosphate-Buffered Saline (PBS) such as Dulbecco’s Phosphate-Buffered Saline (DPBS). The PBS can include a platelet lysate (i.e. 10% PRP lysate of platelet lysate). The SL can then be placed interior side down on a substrate. An entire dissected umbilical cord with the Wharton’s Jelly removed can be placed directly onto the substrate, or the dissected umbilical cord can be cut into smaller sections (e.g. 1-3 mm) and these sections can be placed directly onto the substrate. The substrate can be a solid polymeric material such as a cell culture dish. The SL can be placed upon the substrate of the cell culture dish without any additional pretreatment to the cell culture treated plastic, or on a semi-solid culture medium such as agar. Following placement of the SL on the substrate, the SL is cultured in a suitable medium (e.g. Dulbecco’s Modified Eagle Medium (DMEM) glucose (500-6000 mg/mL) without phenol red, 1 x glutamine, 1 x NEAA, and 0.1-20% PRP lysate or platelet lysate). The culture can then be cultured under either normoxic or hypoxic culture conditions for a period of time sufficient to establish primary cell cultures (e.g. 3-7 days). After primary cell cultures have been established, the SL tissue is removed and discarded. Cells or stem cells are further cultured and expanded in larger culture flasks in either a normoxic or hypoxic culture conditions.

General methods for preparing cell sheets are known in the art and are described, for example, in U.S. Pat. Nos. 8,642,338; 8,889,417; 9,981,064; and 9,114,192, each of which is incorporated by reference herein in its entirety.

The temperature-responsive polymer used to coat the substrate of the cell culture support has an upper or lower critical solution temperature in aqueous solution which is generally in the range of 0° C. to 80° C., for example, 10° C. to 50° C., 0° C. to 50° C., or 20° C. to 45° C.

The temperature-responsive polymer may be a homopolymer or a copolymer. Exemplary polymers are described, for example, in Japanese Patent Laid-Open No. 211865/1990. Specifically, they may be obtained by homo- or co-polymerization of monomers such as, for example, (meth)acrylamide compounds ((meth)acrylamide refers to both acrylamide and methacrylamide), N-(or N,N-di)alkyl-substituted (meth)acrylamide derivatives, and vinyl ether derivatives. In the case of copolymers, any two or more monomers, such as the monomers described above, may be employed. Further, those monomers may be copolymerized with other monomers, one polymer may be grafted to another, two polymers may be copolymerized, or a mixture of polymer and copolymer may be employed. If desired, polymers may be crosslinked to an extent that will not impair their inherent properties.

The substrate which is coated with the polymer may be of any types including those which are commonly used in cell culture, such as glass, modified glass, polystyrene, poly(methyl methacrylate), polyesters, and ceramics.

Methods of coating the support with the temperature-responsive polymer are known in the art and are described, for example, in Japanese Patent Laid-Open No. 211865/1990. Specifically, such coating can be achieved by subjecting the substrate and the above-mentioned monomer or polymer to, for example, electron beam (EB) exposure, irradiation with γ-rays, irradiation with UV rays, plasma treatment, corona treatment, or organic polymerization reaction. Other techniques such as physical adsorption as achieved by coating application and kneading may also be used.

The coverage of the temperature responsive polymer may be in the range of 0.4-3.0 µg/cm², for example, 0.7-2.8 µg/cm², or 0.9-2.5 µg/cm². The morphology of the cell culture support may be, for example, a dish, a multi-plate, a flask or a cell insert.

The cultured cells may be detached and recovered from the cell culture support by adjusting the temperature of the support material to the temperature at which the polymer on the support substrate hydrates, whereupon the cells can be detached. Smooth detachment can be realized by applying a water stream to the gap between the cell sheet and the support. Detachment of the cell sheet may be affected within the culture solution in which the cells have been cultivated or in other isotonic fluids, whichever is suitable. In some embodiments, the hUC-MSCs are cultured in the culture solution on the temperature-responsive polymer for at least 12 hours, at least 24 hours, at least 1 day, at least 2 days, at least 3 days, at least 4 days, at least 5 days, at least 6 days, or at least 7 days before adjusting the temperature of the culture solution to below the lower critical solution temperature for release of the cell sheet from the support material.

In a particular embodiment, the temperature-responsive polymer is poly(N-isopropyl acrylamide) Poly(N-isopropyl acrylamide) has a lower critical solution temperature in water of 31° C. If it is in a free state, it undergoes dehydration in water at temperatures above 31° C. and the polymer chains aggregate to cause turbidity. Conversely, at temperatures of 31° C. and below, the polymer chains hydrate to become dissolved in water, thereby causing release of the cell sheet from the polymer. In a particular embodiment, this polymer covers the surface of a substrate such as a Petri dish and is immobilized on it, for example, by chemical or physical grafting or tethering. Therefore, at temperatures above 31° C., the polymer on the substrate surface also dehydrates but since the polymer chains cover the substrate surface and are immobilized on it, the substrate surface becomes hydrophobic with polymer dehydration. Conversely, at temperatures of 31° C. and below, the polymer on the substrate surface hydrates but since the polymer chains cover the substrate surface and are immobilized on it, the substrate surface becomes hydrophilic with polymer dehydration. The hydrophobic surface is an appropriate surface for the adhesion and growth of cells, whereas the hydrophilic surface inhibits the adhesion of cells and the cells are detached simply by cooling the culture solution.

Culture solutions for mesenchymal stem cells are known in the art and are described, for example, in U.S. Pat. Nos. 9,803,176 and 9,782,439, each of which is incorporated by reference herein in its entirety. In some embodiments, the culture solution comprises human platelet lysate (hPL). In some embodiments, the culture solution comprises fetal bovine serum (FBS). In some embodiments, cell sheets grown in culture solution comprising hPL grow more rapidly to high density, exhibit faster detachment at reduced temperature, weaker cell adhesion, and tend to form cell aggregates even without temperature reduction more readily upon release under poorly controlled sheet production conditions relative to cell sheets grown in culture solution comprising FBS. In some embodiments, cell sheets grown in culture solution comprising hPL secretes higher levels of Human growth factor (HGF) relative to cell sheets grown in culture solution comprising FBS. In some embodiments, cell sheets grown in culture solution comprising hPL have a higher cell density per sheet relative to cell sheets grown in culture solution comprising FBS.

In some embodiments, the culture solution comprises ascorbic acid. In some embodiments, the culture solution is a xeno-free medium, i.e. a medium that may contain products obtained from humans but does not contain products obtained from non-human animals. In some embodiments, the culture solution contains at least one product obtained from a non-human animal (e.g. FBS). In some embodiments, the culture solution does not contain a product obtained from a human. In a particular embodiment, the culture solution comprises one or more of Dulbecco’s Modified Eagle’s Medium (DMEM) (Life Technologies, CA, USA), human platelet lysate (hPL, iBiologics, Phoenix, USA), Glutamax (Life Technologies), MEM Non-Essential Amino Acids Solution (NEAA) (Life Technologies) and an antibiotic, e.g., penicillin streptomycin.

The hUC-MSCs may be passed through one or more subcultures (i.e. passages) prior to culturing the cells in culture solution on a temperature-responsive polymer which has been coated onto a substrate surface of a cell culture support. In some embodiments, the hUC-MSCs are passed through 1, 2, 3, 4, 5, 6, 7, 8, 9, 10, 11, 12, 13, 14 or 15 subcultures prior to culturing the cells in culture solution on a temperature-responsive polymer which has been coated onto a substrate surface of a cell culture support. Any of these values may be used to define a range for the number of subcultures. For example, in some embodiments, the hUC-MSCs are passed through 2 to 10, 4 to 8, or 1 to 12 subcultures prior to culturing the cells on a temperature-responsive polymer. In some embodiments the number of subcultures is less than 1, 2, 3, 4, 5, 6, 7, 8, 9, 10, 11, 12, 13, 14 or 15. In some embodiments, the number of subcultures is at least 1, 2, 3, 4, 5, 6, 7, 8, 9, 10, 11, 12, 13, 14 or 15.

The hUC-MSC sheet may be prepared in a range of different sizes depending on the application. In some embodiments, the hUC-MSC sheet has a diameter of at least 1, 2, 3, 4, 5, 6, 7, 8, 9, 10, 15 or 20 cm. Any of these values may be used to define a range for the size of the hUC-MSC sheet. For example, in some embodiments, the hUC-MSC sheet has a diameter from 1 to 20 cm, from 1 to 10 cm or from 2 to 10 cm. In some embodiments, the hUC-MSC sheet has an area of at least 1, 2, 3, 4, 5, 6, 7, 8, 9, 10, 15, 20, 30, 40, 50, 60, 70, 80, 90, 100, 150, 200, 250 or 300 cm². Any of these values may be used to define a range for the size of the hUC-MSC sheet. For example, in some embodiments, the hUC-MSC sheet has an area from 1 to 100 cm², 3 to 70 cm², or 1 to 300 cm². The methods described herein result in an hUC-MSC sheet in which the surface area of the hUC-MSC sheet is much greater than its thickness. For example, in some embodiments the ratio of the surface area of the hUC-MSC sheet to its thickness is at least 10:1, 100:1, 1000:1, or 10,000:1. The hUC-MSC sheets described herein comprise one or more layers of confluent human umbilical cord mesenchymal stem cells (hUC-MSCs), for example, 1, 2, 3, 4, 5, 6, 7, 8, 9 or 10 layers of hUC-MSCs. In some embodiments, the hUC-MSC sheet comprises fewer than 2, 3, 4, 5, 6, 7, 8, 9 or 10 layers of hUC-MSCs. In some embodiments, the hUC-MSC sheet comprises at least 1, 2, 3, 4, 5, 6, 7, 8, 9 or 10 layers of hUC-MSCs.

### IV. Methods for Transplanting Human Umbilical Cord MSC (hUC-MSC) Sheets to a Subject

The cell sheets described herein can be transplanted to a subject by applying the cell sheet to a tissue in the subject. For example, as disclosed in Example 1 below, when a hUC-MS sheet was prepared by the methods described herein and implanted into dorsal subcutaneous pockets in immune-deficient mice, the cell sheet was stably engrafted to the subcutaneous tissue 10 days after transplantation. In addition, at 10 days after transplantation, capillaries had formed (angiogenesis) in cell sheet-transplanted tissue, while subcutaneous tissue without cell sheet transplantation showed only a few fine blood vessels. Furthermore, in cell sheet-transplanted animals, a large number of blood vessel structures were observed between transplanted cell sheets and host tissue. These results indicate that the cell sheets are transplantable, engraft and preserve cell sheet structures for 10 days in vivo, and induce neocapillary formation as an important capability for engraftment, viability and tissue regeneration.

Accordingly, in some aspects, the present disclosure relates to a method of transplanting a cell sheet to a subject comprising applying a cell sheet as described herein to a tissue of a subject. In a particular embodiment, the subject is a human. One of the advantages of the hUC-MSC sheets described herein is that the extracellular matrix of the cell sheet act as an adhesive to bind the cell sheet to the tissue of the subject, such that stitching is not required to adhere the cell sheet to the tissue. A support membrane may be used to transfer the harvested hUC-MSC sheet released from the culture surface to the tissue of the subject. The support membrane for such transfer can be, for example, poly(vinylidene difluoride) (PVDF), cellulose acetate, and cellulose esters. The hUC-MSC sheets readily adhere to target tissue, self-stabilizing without suturing after being placed directly onto the target tissue for a short period of time. For example, in some embodiments, the hUC-MSC sheet adheres to the target tissue within 5, 10, 15, 20, 25, or 30 minutes after contact with the tissue. Once the hUC-MSC sheet has adhered to the target tissue, the support membrane may be removed or excised. In certain embodiments, the hUC-MSCs in the cell sheet are allogeneic to the subject, i.e. are isolated from a different individual from the same species as the subject, such that the genes at one or more loci are not identical. In certain reported cases, MSCs seemingly avoid allogeneic rejection in humans and in animal models (Jiang et al., 2005, Blood, 105(10), 4120-4126). Thus the hUC-MSC sheets described herein may be used in allogeneic cell therapies as an off-the-shelf product, avoiding the unfavorable costs and development disincentives associated with autologous stem cell treatment methods.

Allogeneic cell sources must be capable of eliciting meaningful therapies under standard immunologic competence in host patient allogeneic tissues. This includes reliable cell homing to and fractional dose engraftment or retention for sufficient duration at the tissue site of therapeutic interest (Leor et al., 2000, Circulation, 102(19 Suppl 3), III 56-61). Current estimates are that when stem cell suspensions are administered to a subject, less than 3% of injected stem cells are retained in damaged myocardium 3 days post-injection following ischemic injury (Devine et al., 2003, Blood, 101(8), 2999-3001). Additionally, most administered cells from a cell suspension that engraft into target tissue will die within the first few weeks (Reinecke & Murry, 2002, J Mol Cell Cardiol, 34(3), 251-253). In contrast, as discussed above, the hUC-MSC cell sheets described herein were stably engrafted to the subcutaneous tissue 10 days after transplantation. Thus the hUC-MSC sheets described herein provide distinct advantages over mesenchymal stem cell suspensions.

### EXAMPLES

### Example 1. Properties of Umbilical Cord Mesenchymal Stem Cell Sheets Prepared in Xeno-Free Media

### Materials and Methods

### 1.1 Human Umbilical Cord Stem Cell (hUC-MSC) Culture

Banked human umbilical cord mesenchymal stem cells isolated from the subepithelial layer of human umbilical cord tissue (Jadi Cell LLC, Miami, USA IRB-35242) (Patel et al., 2013, Cell Transplant, 22(3), 513-519) were cultured in xeno-free cell culture media with Dulbecco’s Modified Eagle’s Medium (DMEM) (Life Technologies, CA, USA) supplemented with 10% human platelet lysate (hPL, iBiologics, Phoenix, USA), 1% Glutamax (Life Technologies), 1% MEM NEAA (Life Technologies), 1% penicillin streptomycin (Life Technologies), at 37° C. in a humidified atmosphere with 5% CO₂ for 5 days. Subculture was performed from passage 4 until passage 12. Cell culture media was changed every two days.

### 1.2 hUC-MSC Proliferation Rate

hUC-MSCs were seeded on 35-mm tissue culture plates (TCP) (Corning, NY) at cell numbers of 5×10^{4,} 1×10⁵ and 2×10⁵ cells/dish (i.e. initial cell densities of 5×10³/cm², 1×10⁴/cm², and 2×10⁴/cm², respectively) in xeno-free cell culture media. Cells on TCP were dissociated with trypsin and cell number counted using a hemocytometer at 1, 2, 3, 4, 5 and 6 days. hUC-MSCs were seeded at a cell density of 3.5×10³/cm² on 175 cm² tissue culture flasks (Corning, NY) and passaged at 5 days with TrypLE (life technologies) after culturing from passage 4 until 12. Cell number was counted each passage using a hemocytometer.

### 1.3 hUC-MSC Characterization in Differentiation Potential

hUC-MSCs were cultured in xeno-free cell culture media for two passages on TCP. At passages 4, 6, 8, 10, and 12, cells were prepared and induced for osteogenic and adipogenic differentiation. For osteogenic differentiation, cells were plated at 5×10³ cells/cm² in 35 mm TCP dishes in xeno-free cell culture media. When 60% confluent, cells were induced with osteogenic differentiation media containing αMEM, 10 nM dexamethasone, 82 µg/mL ascorbic acid 2-phosphate, 10 mM β-glycerolphosphate (Sigma-Aldrich). Cells were cultured in osteogenic media at 37° C. for 21 days with media changed every 3 days. To detect positive differentiation, cells were fixed with cold 4% paraformaldehyde for 12 minutes and stained with Alizarin Red S-(Sigma-Aldrich) using standard protocols. For adipogenic differentiation, cells were plated at 1×10⁴ cells/cm² in 35 mm TCP dishes in xeno-free cell culture media. When 80% confluent, cells were induced with adipogenic differentiation media containing high-glucose DMEM, 100 nM dexamethasone, 0.5 mM IBMX, and 50 µM IND (all Sigma-Aldrich). Cells were cultured in adipogenic media at 37° C. for 21 days and media changed every 3 days. To detect positive differentiation, cells were fixed with cold 4% paraformaldehyde for 12 minutes and stained with Oil Red O (Sigma-Aldrich) using standard protocols.

### 1.4 hUC-MSC Surface Phenotype Assay

hUC-MSCs were cultured in xeno-free cell culture media on TCP. Cell suspensions were prepared of P6, P8, P10, and P12 HPL and FBS cultured cells. Cells were then detached enzymatically and washed once with PBS. To minimize non-specific binding of antibodies, cells were incubated with 2% w/v Bovine Serum Albumin (BSA) in PBS for 30 minutes. Cells were then aliquoted at concentrations of 3-5 × 10⁵/100 µL. One aliquot was reserved as an unstained control and those remaining were stained with the following antibodies: CD44, CD90 and HLA-DR,DP,DQ (Biolegend, San Diego, CA). Primary antibody was added to each aliquot to achieve a ratio of about 20:1 of cells in buffer to antibody. About 3-5 × 10⁵ cells were stained with saturating concentrations of (fluorophore)-conjugated antibodies. Cells were incubated in the dark on ice for 30 minutes. After incubation, cells were washed three times and then resuspended in PBS. The cells were immediately analyzed by flow cytometry. Flow cytometry was performed on a Becton, Dickinson FACS Canto (BD Biosciences, Sparks, MD). Flow cytometer instruments were set using unstained cells. Cells were gated by forward versus side scatter to eliminate doublets. A minimum of 10,000 events was counted for each analysis.

### 1.5 hUC-MSC Sheet Preparation Using Different Initial Cell Numbers and Passage Numbers

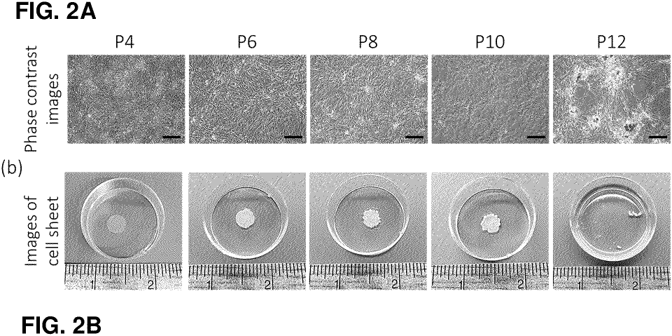

hUC-MSC sheets were prepared on temperature-responsive cell culture dishes (TRCDs) in various conditions including different initial cell density and passage numbers (FIG. 2). Passage 6 cells were seeded on 35-mm TRCDs (CellSeed Inc., Tokyo, Japan) at cell numbers of 5×10⁴ cells/dish, 1×10⁵ cells/dish and 2×10⁵ cells/dish. Passage 4-12 cells were seeded at a cell number of 2×10⁵ cells/dish (i.e. an initial cell density of 2×10⁴/cm²). Fresh xeno-free cell culture media including 16.4 µg/mL of ascorbic acid (Sigma-Aldrich, St. Louis, USA) to make cell sheets was added at 1 day after seeding. Confluent cell sheets formed at 4-6 days after seeding and were detached from TRCD at room temperature. Cell morphologies were monitored using an AX 10 microscope (Carl Zeiss Microimaging, Göttingen, Germany) with Axio Vision software (Carl Zeiss Microimaging) before cell sheets detachment.

### 1.6 Immunohistochemical Staining

Cultured cell sheets were removed from TRCD at room temperature and fixed with 4% paraformaldehyde for 30 min and then embedded in paraffin. Embedded specimens were sectioned into 4 µm slices and stained with H&E, stem cell surface markers, ECMs (fibronectin; FN and laminin; LM) and cell-cell junctions (integrin-linked kinase; β-catenin). For fluorescence staining (FN, LM, and β-catenin), slides were immersed in antigen retriever solution (Sigma-Aldrich) for 20 min at 100° C. and washed with PBS 1X. Non-specific binding was blocked in PBS 1X containing 10% goat serum (Vector Laboratories, Burlingame, USA), for 1 h at room temperature. Primary antibody labeling (Abcam, Cambridge, USA) (1:100) at 4° C. proceeded overnight and then washed with PBS 1X. These specimens were treated with Alexa Fluor 594-conjugated secondary antibodies (Life Technologies) (1:200) for 1 h and mounted with ProLong Gold Antifade Reagent (Life Technologies). Immunofluorescence images were obtained using an AX 10 microscope (Carl Zeiss Microimaging) and analyzed with Axiovision software (Carl Zeiss Microimaging). For H&E stain, specimens were treated with hematoxylin solution (Sigma-Aldrich) for 3 min and subsequently with eosin solution (Thermo Fisher Scientific, Kalamazoo, USA) for 5 min. The H&E stained specimens were dehydrated and mounted with Permount™ (Thermo Fisher Scientific). H&E images were obtained using a BX 41 microscope (Olympus, Hamburg, Germany).

### 1.7 Cell Sheet Microstructure Observed Using Transmission Electron Microscopy

hUC-MSC sheets were fixed with a mixture of 2% paraformaldehyde, 2% glutaraldehyde, 0.1 M sodium phosphate buffer, and 2% osmium tetroxide (OsO₄) in sodium phosphate buffer and dehydrated in a grade series of ethanol. Samples were then embedded in epoxy resin. Ultrathin sections (70 nm thickness) were observed with a transmission electron microscope (JEOL JEM1200EX) (JEOL USA, Peabody, USA).

### 1.8 Determination of Hepatocyte Growth Factor (HGF) and Tumor Necrosis Factor Alpha (TNF-α) Secretion From hUC-MSC Sheets

hUC-MSC cell sheets were fabricated on TRCDs. Supernatant media over adherent cultured cells for 24 hours was collected just prior to cell sheet detachment from TRCD at room temperature (RT). HGF and TNF-α amounts secreted from hUC-MSCs were measured by human HGF Quantikine ELISA and human TNF-α Quantikine ELISA kits, respectively (R&D Systems, Minneapolis, USA).

### 1.9 Cell Sheet Placement Into Immune-Deficient Mice Subcutaneous Tissue

hUC-MSC (passage 6) cell sheets were detached from TRCD at RT after 4 days of culture and transplanted into subcutaneous dorsal tissues of 6-week old immune-deficient mice (NOD.CB 17-Prkdc^{scid}/NCrCrl) (Charles River, San Diego, USA). Sterilized non-cytotoxic silicone membrane (Invitrogen) was placed between the cell sheet and subcutaneous dorsal tissues to prohibit tissue adhesion. Implanted mice were sacrificed 10 days after cell sheet transplantation. The cell sheet-transplanted subcutaneous tissue was fixed with 10% paraformaldehyde (Sigma-Aldrich) for 1 day for histological analysis (see Materials and Methods; 2.6. Immunohistochemical staining). All procedures were approved by the Institutional Animal Care and Use Committee (IACUC) (protocol #16-12017) at The University of Utah and conducted in accordance with national guidelines.

### 1.10 Statistical Analyses

All quantitative values are expressed as mean and standard error (SE, mean±SE). Significant differences between groups were tested by one-way Analysis of Variance using origin 2017 software (OriginLab, Northampton, USA). A probability value of less than 0.05 (p < 0.05) was considered statistically significant.

### 1.11 Quantitative Reverse Transcription Polymerase Chain Reaction (RT-PCR) Analysis

hUC-MSC cell sheets were collected after detachment from TRCD at RT. Total RNA from cell sheets was extracted using trizol and PureLink RNA Mini Kt (Life Technologies) according to manufacturer’s protocols. cDNA was prepared from 1 µg of total RNA using high capacity cDNA reverse transcription kits (Life Technologies). RT-PCR analysis was performed with TapMan Universal PCR Master Mix using an Applied Biosystems Step One instrument (Applied Biosystems™, Foster City, USA). Gene expression levels were assessed for the following genes: 1) glyceraldehyde 3-phosphate dehydrogenase (GAPDH, Hs02786624_gl) as a housekeeping gene, 2) integrin-linked kinase (ILK, Hs00177914_m1), 3) N-cadherin (N-cad, Hs00983056_m1). All primers were manufactured by Applied Biosystems (sequences for each shown in Table S1, Supplementary data). Relative gene expression levels were quantified by the comparative C_{T} method (Schmittgen & Livak, 2008). Gene expression levels were normalized to GAPDH expression levels. Gene expression levels are relative to the level at passage 6 cell group.

### Results

### hUC-MSC Sheet Preparation With Different Initial Cell Numbers and Passage Numbers

hUC-MSCs were cultured on flasks and sub-cultured using trypsin every 5 days from passages 4 to 12 (Table 1). Cells were proliferated 16-20 times from initial cell seeding numbers between passages 4-8 during sub-culture. However, cell proliferation rate dramatically decreases from passage 9. Cell numbers were 14, 10.9, 7.5, and 3.1-fold increased from initial cell seeding numbers at passage 9, 10, 11, and 12, respectively. Cells in passage 10 required one day more to reach confluence and yield cell sheets than cells in passages 4-8 at the same seeding density (FIGS. 2a and b). Cells in passage 12 exhibited heterogeneously cultured morphologies, lost contact inhibition, and clumped into multi-layered aggregates rather than consistent monolayers (FIG. 2a). When culture temperature was reduced to room temperature (RT), cells in passage 12 detached from TRCD, but not as sheets, as recovered from passages 4-10 (FIG. 2b). Cells should therefore be used from passages 4 to 8 to produce consistent cell sheet quality.

TABLE 1

Growth rates for hUC-MSCs in passages 4-12

| | Cell viability | Initial cell seeding density | Expended cell density | Fold increase |
|---|---|---|---|---|
| Passage 4 | 96% | 4×10³/cm² | 5.5×10⁴/cm² | 16.3 |
| Passage 5 | 96% | 3.5×10³/cm² | 5.7×10⁴/cm² | 19 |
| Passage 6 | 97.5% | 3.2×10³/cm² | 5.5×10⁴/cm² | 17.2 |
| Passage 7 | 96.2% | 3.2×10³/cm² | 5.5×10⁴/cm² | 17.3 |
| Passage 8 | 96.7% | 3.2×10³/cm² | 6.6×10⁴/cm² | 20.7 |
| Passage 9 | 98.5% | 3.2×10³/cm² | 4.5×10⁴/cm² | 14 |
| Passage 10 | 97.6% | 3.2×10³/cm² | 3.5×10⁴/cm² | 10.9 |
| Passage 11 | 97.4% | 3.2×10³/cm² | 2.4×10⁴/cm² | 7.5 |
| Passage 12 | 97.4% | 3.2×10³/cm² | 1.0×10⁴/cm² | 3 1 |

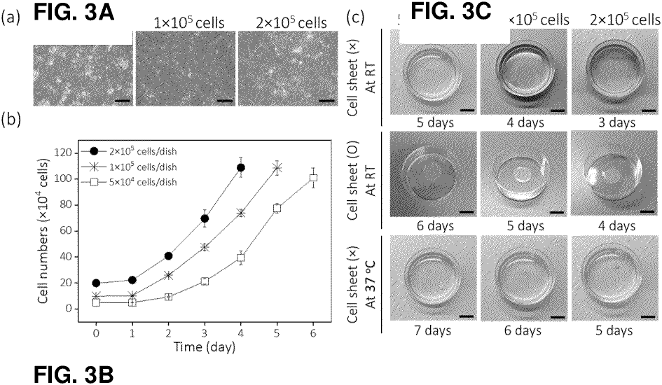

Initially seeded cell numbers of 5×10^{4,} 1×10⁵, and 2×10⁵ cells/dish reached confluence at 6, 5, and 4 days, respectively (FIGS. 3a and b), reliably producing hUC-MSC sheets (FIG. 3c). Cell sheets from all different initial cell number groups spontaneously detached from TRCDs without temperature change (i.e., at 37° C.) at 5, 6, and 7 days in the 2×10⁵, 1×10⁵, and 5×10⁴ seeded cells/dish groups, respectively (FIG. 3c) once cells were over-confluent. One day prior to cell confluence, adherent cells were less than 70-80% confluent and detached as partially broken sheet fragments when temperature was reduced to RT due to insufficient cell density. Recovery of hUC-MSC sheets either one day before or one day after cells reaching confluence on TRCDs was not possible (FIG. 3c). These results indicate that cell sheets must be prepared carefully and recovered at the precise time-point when cells are neither under- or over-confluent in order to detach the cells as a thermally recovered sheet.

### hUC-MSC Surface Marker Characterization

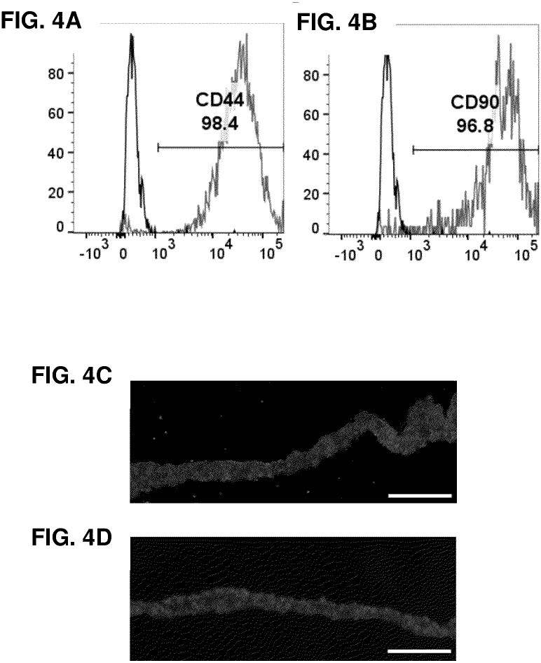

CD44 and CD90 are known to be expressed in hUC-MSCs. CD44 and CD90 expression was measured in hUC-MSC suspension cultures and in hUC-MSC sheets in vitro. As shown in FIG. 4, hUC-MSCs expressed CD44 and CD90 in suspension cultures (FIGS. 4A and 4B) and in cell sheets (FIGS. 4C and 4D) in vitro. CD44 and CD90 are known to be expressed in hUC-MSCs. Accordingly, these results indicate that hUC-MSC sheets maintained hUC-MSC specific phenotypes. In particular, the results in FIGS. 4C and 4D indicate that the cell sheets contain hUC-MSCs and do not contain differentiated cells and other cell types from the umbilical cord.

### Structural Analysis of hUC-MSC Sheets

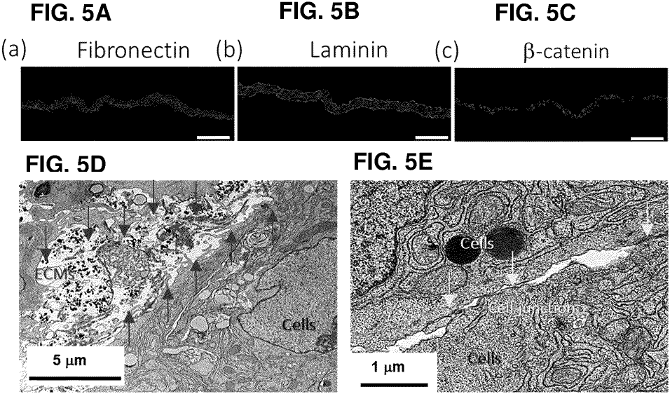

Passage 6 cells were cultured on TRCD for 4 days and resulting cell sheets were recovered from TRCD with temperature reduction to RT. The cell sheet was stained with fibronectin, laminin, and β-catenin to verify hUC-MSC sheet retention of functional structures during culture and after sheet detachment. Fibronectin and laminin, important ECM components that promote cell and tissue attachment (Yue, 2014, J Glaucoma 23: S20-S23; Kim et al., 2016, Int Neurourol J.: S23-S29), were strongly expressed across the entire cell sheet surface (FIGS. 5a and b). β-catenin, part of the protein complex forming cell adherent junctions(Nelson & Nusse, 2004, Science, 303(5663), 1483-1487), shows prominent staining between cells (FIG. 5c). Retention of ECM and cell junction proteins indicates that functional proteins produced during culture are preserved after cell sheet harvest.

Inter-cellular structures within cell sheets were observed by TEM. Horizontal sectioning showed ECM structures within cell sheets (FIG. 5d), including numerous cell-cell junctions (FIG. 5e). These results suggest that hUC-MSC sheets structurally retain functional proteins related to natural cell functions such as cell communication and cell adhesion.

### Secretion of Hepatocyte Growth Factor (HGF) and Tumor Necrosis Factor-Alpha (TNF-α)

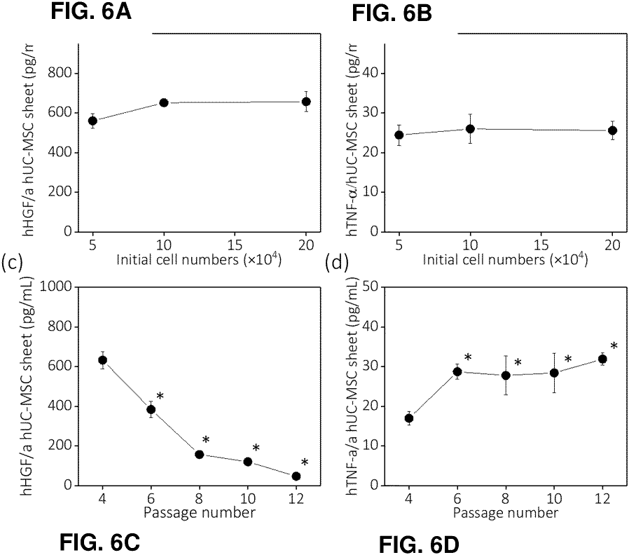

Human anti-inflammatory cytokine HGF (Gong, Rifai, & Dworkin, 2006; J Am Soc Nephrol, 17(9), 2464-2473), and pro-inflammatory cytokine TNF-α (Ertel et al., 1995, J Cell Sci, 123(Pt 24), 4195-4200) (REF) secreted from hUC-MSCs in culture supernatant were measured to support paracrine effects of the fabricated hUC-MSC sheets in vitro. No significant differences in amounts of hHGF were seen in 2×10⁵, 1×10⁵, and 5×10⁴ cells/dish groups at passage 6 (FIG. 6a). Pro-inflammatory cytokine (hTNF-a) was barely detectable in 2×10⁵, 1×10⁵, and 5×10⁴ cells/dish groups (FIG. 6b).

hUC-MSC sheets fabricated using passage 4 cells secreted significantly higher concentrations of hHGF (633 pg/mL), compared to hUC-MSC sheets fabricated using passage 6, 8, 10, and 12 cells. Amounts of hHGF secreted from hUC-MSC sheets dramatically decreased as passage number increased (FIG. 6c). hTNF-α was barely secreted (16-35 pg/mL) from hUC-MSC sheets (FIG. 6d) and hUC-MSC sheets fabricated using passage 4 had significantly lower concentrations of hTNF-α, compared to hUC-MSC sheets fabricated using passage 6, 8, 10, and 12 cells. Results therefore demonstrate that passage number is an important factor in hUC-MSC sheet cytokine properties.

### Cell Sheet Implantation Into Immune-Deficient Mice

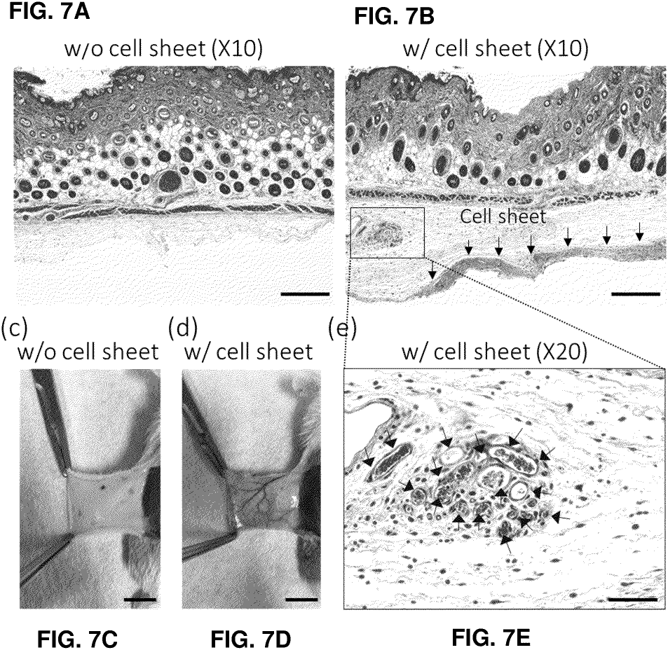

hUC-MSC sheets were implanted into dorsal subcutaneous pockets in immune-deficient mice for 10 days to demonstrate stability and engraftment in vivo. At 10 days after transplantation, formation of capillaries (angiogenesis) was observed in cell sheet-transplanted tissue, while subcutaneous tissue without cell sheet transplantation showed only a few fine blood vessels (FIGS. 7c and d). H&E staining data demonstrated that cell sheets remained localized on the transplanted area for 10 days after transplantation (FIGS. 7a and b). In cell sheet-transplanted groups, a large number of blood vessel structures was observed between transplanted cell sheets and host tissue (FIG. 7e). This indicates that the cell sheets are transplantable, engraft and preserve cell sheet structures for 10 days in vivo. Furthermore, cell sheets induce neocapillary formation as an important capability for engraftment, viability and tissue regeneration.

### Discussion

Xeno-free hUC-MSC sheet fabrication was demonstrated from cultures using temperature responsive culture dishes (TRCD). These hUC-MSC sheets exhibit: 1) retention of native functional inter-cellular structures essential to cell-cell communication, act as a natural matrix adhesive when implanted onto target organs (FIG. 5); 2) hepatocyte growth factor (HGF) secretion inducing angiogenesis and anti-fibrotic action (FIG. 6); 3) cell retention in vivo for 10 days after implantation; and 4) vascular neogenesis in vivo supporting sheet-tissue engraftment (FIG. 7).

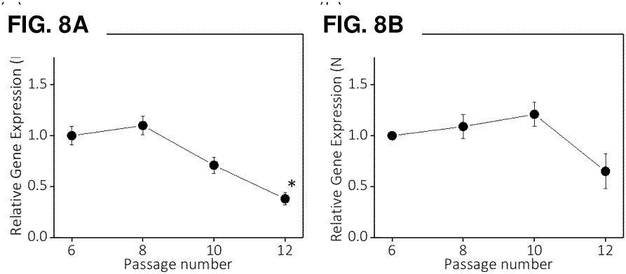

To fabricate potent MSC cell sheets reproducibly, hUC-MSCs from passages 4 to 12 were expanded and transformed to sheets in cell culture media supplemented with hPL. Cell proliferation rates for hUC-MSCs were remarkably reduced after passage 10, affecting the cell sheet creation process and timelines to harvest (FIG. 2). Furthermore, passage 12 cells were not able to form stable sheets due to reduced cell proliferation rates and inadequate cell-cell junction formation after increased passaging (Table 1 and FIG. 8). In addition, microscopy phase contrast images (FIG. 2) showed cells stacked on top of each other and formation of cell aggregates at higher passage numbers. This feature tends to increase as passage number increases, especially for passage 12 cells. Cell aggregations using bone marrow derived (BMSCs) and adipose derived (ADSC) stem cells is reported to occur when cultured in media including 5% hPL (Hemeda, Giebel, & Wagner, 2014, Cytotherapy, 16(2), 170-180). Active coagulation factors in hPL could be involved in aggregation. This cell aggregations interrupts homogeneous cell growth and cell sheet fabrication. To prepare reproducible hUC-MSC sheets, passage numbers below passage 10 would be preferable.

Rapid growth of hUC-MSCs cultured in cell culture media including hPL could be beneficial in reducing time required to fabricate cell sheets. Conversely, it may also introduce some processing difficulties because sheet cultures reach confluence quickly and are prone to spontaneous detachment upon reaching confluence. Therefore, judicious use of appropriate initial cell seeding numbers is important for the hUC-MSC sheet fabrication process. Initial cell seeding density higher than 2×10⁵ cells/dish does not yield a monolayer sheet: such high density induces spontaneous cell detachment from TRCDs within 2 days of cell culture (data not shown). In this study, initial densities of 2×10⁵, 1×10⁵, and 5×10⁴ cells were used and all successfully yielded hUC-MSC sheets at day 4, 5, and 6, respectively, when each culture reached confluence (FIG. 3). Poor cell sheet quality was observed when cells were detached one day prior to or one day after reaching confluence (FIG. 3c). Therefore, for best hUC-MSC sheet fabrication, the harvest time for TRCD cell sheet recovery depends on both passage number and seeding cell density.

Central to these results is the reliable capability to produce a stable, robust monolayer of hUC-MSCs using a commercial TRCD grafted with temperature-responsive polymer coating that facilitates cell harvest without destructive enzymes using temperature reduction (Okano et al., 1995, Biomaterials, 16(4), 297-303; Okano et al., 1993, J Biomed Mater Res, 27(10), 1243-1251). This cell sheet technology produces cultured cell recovery with intact native cell-cell organization, cell-cell communication, intact ECMs, and tissue-like phenotypes. Cell sheets recovered from TRCDs by small changes in culture temperature preserve cell surface-associated ECMs such as fibronectin and laminin, and cell-cell junction proteins such as β-catenin (FIG. 5), that play important roles in promoting cell adhesion and paracrine signaling (Yue, 2014, J Glaucoma 23: S20-S23; Kim et al., 2016, Int Neurourol J.: S23-S29). Cell sheets with native morphologies, confluent phenotypes and organization, cell-cell communications, intact extracellular matrix (ECM) and tissue-like behaviors can be readily transferred to target tissues (Miyahara et al., 2006, Nat Med, 12(4), 459-465. hUC-MSC sheets implanted into subcutaneous tissue sites in immune-deficient mice rapidly and spontaneously attached to subcutaneous tissue surfaces within 10 min. After 10 days in vivo, implanted cell sheets remained as intact sheets (FIG. 7).

Overall, hUC-MSC sheets display several beneficial properties for improving allogeneic MSC cell therapy. Results here have determined (1) specific conditions for reliable xeno-free hUC-MSC sheet fabrication; (2) intact features of hUC-MSC sheets that preserve important cell functional structures and paracrine effects after cell harvest from TRCDs; (3) intact hUC-MSC sheet retention in implant target tissue sites for 10 days; and (4) new blood vessel recruitment into sheets on the target tissue, suggesting that implanted hUC-MSC sheets continually secret paracrine factors to modulate engraftment.

### Conclusions

hUC-MSC cell sheet technology represents a unique cellular delivery method aimed to improve MSC therapy over current injected cell suspensions. The simple fabrication method on TRCDs in hPL allows rapid xeno-free production of robust uniform monolayer hUC-MSC sheets, harvested with small changes of temperature instead of destructive proteolytic enzymes. Cell production depends on several controlled culture variables, including cell seeding density, passage number, media (hPL), and culture time and TRCDs. When cultured homogeneously under optimized conditions, hUC-MSC cell sheet reproducibility is enhanced and the hUC-MSC cell sheet production process is simplified to a routine amenable to scaling. This enables future production of hUC-MSC sheets having higher cell numbers to increase paracrine action and therapeutic benefits. Given their paracrine effects and low HLA profile, fabricated xeno-free hUC-MSC sheets represent promising tissue regeneration potential both structurally and functionally in vitro and in vivo. With reliable topical tissue site placement, high engraftment efficiency, long-term retention and survival in vivo, the hUC-MSC sheet has a potential to improve therapeutic value of allogeneic cell therapy over injected stem cells used currently.

### Example 2. Comparison of Human Umbilical Cord Mesenchymal Stem Cells (hUC-MSCs) Harvested by Temperature Change, Trypsin Treatment, and Cell Scraper

### Materials and Methods

### 2.1 Antibodies

The following antibodies were used in this study; actin (ab8226) (Abcam, Cambridge, USA), vinculin (ab129002) (Abcam), fibronectin (ab6328) (Abcam), laminin (ab11575) (Abcam), integrin β-1 (ab179471) (Abcam), connexin 43 / GJA1 (ab11370) (Abcam), YAP (#140794) (Cell Signaling Technology (CST), Massachusetts, USA), phospho-YAP (Ser127, #4911)) (CST), FAK (ab40794) (Abcam), Phospho-FAK (Tyr397, #8556) (CST), GAPDH (ab9484) (Abcam). Alexa flour 568 goat anti-rabbit, 568 goat anti-mouse, 488 goat anti-rabbit, and 488 goat anti-mouse (life technologies) were used as secondary antibodies.

### 2.2 Human Umbilical Cord Stem Cell (hUC-MSC) Culture

Banked human umbilical cord mesenchymal stem cells (hUC-MSCs) were isolated from the subepithelial layer of human umbilical cord tissue (Jadi Cell LLC, Miami, USA IRB-35242) and were cultured in Dulbecco’s Modified Eagle’s Medium (DMEM) (Gibco, Massachusetts, USA) supplemented with 10% fetal bovine serum (FBS) (Gibco), 1% GlutaMAX (Gibco), 1% MEM non-essential amino acids (NEAA) (Gibco), 100 units/mL penicillin, and 100 µg/mL streptomycin (Gibco). hUC-MSC was incubated at 37° C. with 5% CO₂ in a humidified chamber and passaged when cells reached confluent. hUC-MSC was passaged with TrypLE (Gibco) treatment for 5 minutes and subculture at 3000 cells/cm² between passages 4 and 6.

### 2.3 Preparation of hUC-MSC Sheet

hUC-MSCs were seeded on a 35 mm temperature responsive culture dish (TRCD) (CellSeed, Tokyo, Japan). hUC-MSC was seeded at the density of 2 × 10⁵ cells/dish (Day 0) and cultured to confluence (Day 5). Cell culture media including 16.4 µg/mL of ascorbic acid (Wako, Osaka, Japan) was replaced at 1 day after seeding. hUC-MSC was harvested as a mono-layer sheet from TRCD within 60 minutes by reducing the temperature to 20° C. Total cell number of hUC-MSC sheet was counted with trypan blue (Gibco) exclusion test using hemocytometer.

### 2.4 Hematoxylin and Eosin (H&E) Staining of hUC-MSC Sheet

Samples were fixed with 4% buffered paraformaldehyde (PFA) and embedded in paraffin. Then, the samples were cut into 4 µm-thick sections. The sections were stained with mayer’s hematoxylin and 1% eosin alcohol solution. Then, it was mounted with permount™ (Thermo Fisher Scientific). The stained samples were visualized using a BX53 microscope (Olympus, Tokyo).

### 2.5 Morphological Observation of hUC-MSCs Using Scanning Electron Microscopy and Transmission Electron Microscope

For scanning electron microscopy (SEM) analysis, samples were rinsed in wash buffer (0.1 M sodium cacodylate buffer with 2.4% sucrose and 8 mM calcium chloride) for 5 minutes and then fixed with 2% osmium tetroxide (OsO₄) in wash buffer for 1 hour at room temperature. Samples were rinsed with DI water to remove unbound osmium, then dehydrated through grade series of ethanol. Subsequently, ethanol was replaced with hexamethyldisilazane (HMDS) and dried at -30° C. The samples were observed with scanning electron microscope (FEI Quanta 600 FEG, FEI, Oregon). For transmission electron microscope (TEM) analysis, samples were fixed with a mixture of 2% paraformaldehyde, 2% glutaraldehyde, 2% OsO4 in 0.1 M sodium phosphate buffer and dehydrated in grade series of ethanol. The samples were then embedded in epoxy resin to cut into 70 nm thickness. The ultrathin sections were observed with a transmission electron microscope (JEOL JEM-1400 Plus, JEOL, Tokyo).

### 2.6 Cell Viability Assay

Cell viability was measured with live and dead viability/cytotoxicity assay (Thermo Fisher Scientific, MA). Cell sheet and trypsin treated cell groups were washed twice and incubated with Live/Dead working solution (4 mM ethidium homodimer-1 and 2 mM calcein AM) for 30 minutes at 37° C. in the dark. The samples were washed with and visualized using an AX 10 microscope (Carl Zeiss Microimaging, Göttingen, Germany) and analyzed with Axiovision software (Carl Zeiss Microimaging) (Ex / Em 495/635 ethidium homodimer- 1; Ex/Em 495/515 calcein). The number of live and dead cells in single suspension group were counted using image J (National Institutes of Health, Bethesda, Maryland, USA). The number of dead cells in cell sheet was also counted using image J (National Institutes of Health), whereas live cells in cell sheet was calculated based following;

$$\begin{matrix}N u m b e r o f l i v e c e l l s i n 1 s a m p l e = \\ \frac{A r e a o f 1 s a m p l e \left(c {m}^{2}\right)}{T o t a l a r e a o f c e l l s h e e t \left(c {m}^{2}\right)} \times T o t a l c e l l n u m b e r\end{matrix}$$

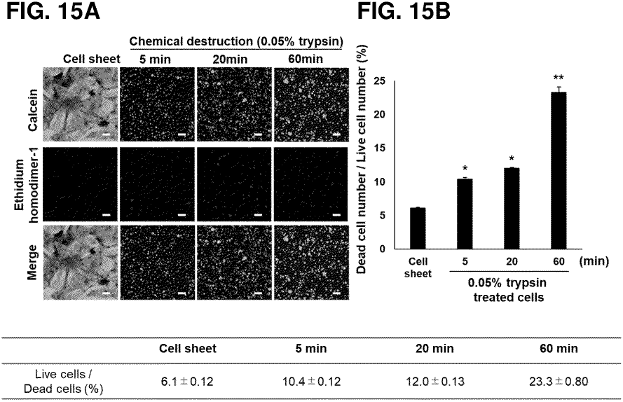

As shown in FIG. 15B, the ratio of dead cells was calculated to compare cell survival rate in each sample.

### 2.7 Qualitative Analysis of Proteins Related to Cell Functions

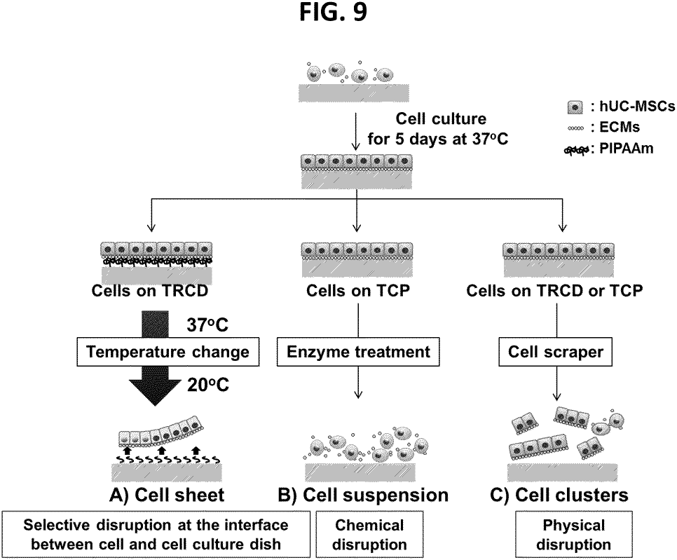

hUC-MSCs (2 ×10⁵ cells/dish) were cultured for 5 days and harvested by temperature change (cell sheet technology), trypsin treatment (chemical disruption), or cell scraper (physical disruption) (FIG. 9). Cells were lysed with cell lysis buffer (RIPA buffer, proteinase inhibitor and phosphatase inhibitor) (Thermo Fisher Scientific) for 15 minutes at 4° C. to isolate protein extracts. Samples were then sonicated for 9 sec three times. The protein concentration of each sample was determined by bradford method (Galipeau et al., 2018, Cell Stem Cell 22(6): 824-833). The samples containing same amount of proteins (10 µg) were denatured at 70° C. for 10 minutes and were loaded onto SDS-PAGE gel (3-8% tris-acetate gels or 4-12% tris-glycine gel (Thermo Fisher Scientific)) and transferred electrophoretically to PVDF membranes (LC2002) (Thermo Fisher Scientific). The membranes were treated with blocking solution 5% bovine serum albumin (BSA) for 1 hour at room temperature and incubated with primary antibodies at 4° C. overnight; actin (1:1000 dilution), vinculin (1:10000 dilution), fibronectin (1:2000 dilution), laminin (1:1000 dilution), integrin β-1 (1:2000 dilution), connexin 43 (1:8000 dilution), YAP (1:1000 dilution), phosphor-YAP (Ser127) (1:1000 dilution), FAK (1:1000 dilution), phospho -FAK (Tyr397) (1:1000 dilution), GAPDH (1:5000 dilution). The incubated membranes were treated with appropriate HRP-conjugated secondary antibodies at room temperature for 1 hour. The membrane was visualized by using enhanced chemiluminescence (FluorChem HD2, ProteinSimple, California, USA). The expression levels were normalized to GAPDH.

### 2.8 Immunocytochemistry Staining of Proteins Related to Cell Functions

Samples were fixed in 4% buffered PFA and then permeabilized with 0.1% triton X-100 (Thermo Fisher Scientific). The samples were blocked with 1% BSA in 10% goat serum for 15 minutes and then incubated in primary antibodies overnight at 4° C.; actin (5 µg/ml), vinculin (1:50 dilution), fibronectin (1:100 dilution), laminin (1:50 dilution), collagen-1 (1:100 dilution), integrin β-1 (1:200 dilution), connexin 43 (1:100 dilution) in the presence of 1% BSA with 10% goat serum. The samples were treated with secondary antibodies for 1 hour. Finally, it was mounted with mounting solution (ProLong Gold Antifade Mountant with DAPI) (Thermo Fisher Scientific) and inspected using IX73 fluorescence microscope (Olympus).

### 2. 9 Statistical Analysis

All values are expressed as the mean ± SEM. Two-way analysis of variance followed by the Tukey’s test was used to evaluate differences between more than two groups. Probabilities (p<0.1, 0.05) were considered significant.

### Results

### Human Umbilical Cord Stem Cell (hUC-MSC) Sheet Preparation

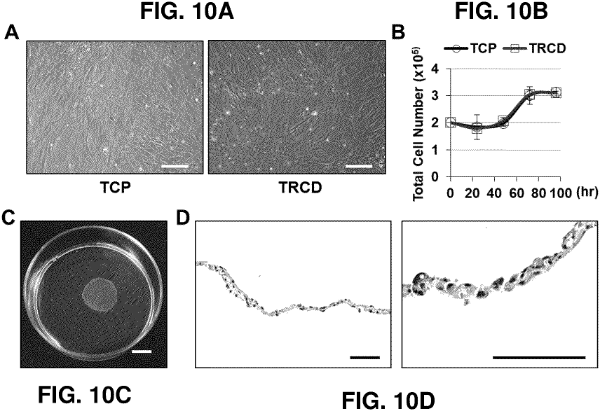

To verify morphologies and growth rate of hUC-MSCs cultured on temperature responsive cell culture dish (TRCD), hUC-MSCs were seeded at the density of 2 × 10⁵ cells on conventional tissue culture plate (TCP) or on 35 mm TRCD and were cultured for 5 days. Cells cultured on TRCD have changed its morphology from rounded shape to spindle shape when cells attached to the bottom surface of TRCD. This morphological change was also observed in cells cultured with TCP (FIG. 10A). Additionally, the growth rate of hUC-MSCs cultured on TRCD showed same growth curve with that on TCP (FIG. 10B). This indicates that the cell culture dish surface coated with temperature responsive polymer didn’t affect growth and morphologies of cells. Furthermore, the cells were successfully detached maintaining a sheet form TRCD with temperature decrease from 37° C. to 20° C. (FIG. 10C). The fabricated cell sheet formed a monolayer and maintained cell binding proteins like as native structures (FIG. 10D).

### Morphological Observation of hUC-MSC Sheet

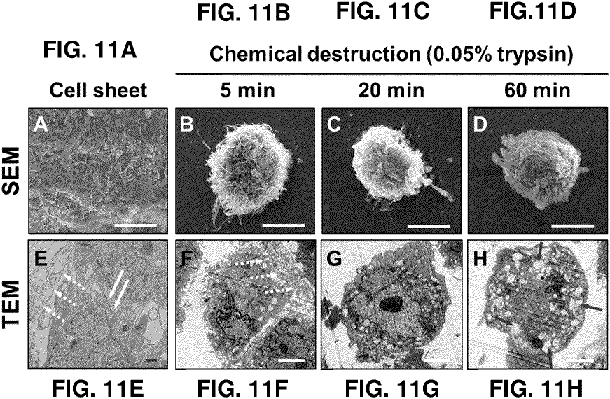

The surface and intercellular structures of hUC-MSC sheets were observed by scanning electron microscopy (SEM) (FIGS. 11A-D) and transmission electron microscopy (TEM) (FIGS. 11E-F). In SEM analysis, hUC-MSC sheet showed connected cell membrane structures on the cell surface. It means that hUC-MSC sheet preserved native structures formed when they cultured on cell culture dishes even after cell detachment. Native cellular membrane structure is comprised of cell surface proteins and membrane proteins, which is related to cell adhesion and functions. This finding suggests that hUC-MSC sheet retaining cell surface proteins and membrane proteins may improve cell adhesion and functions (Albuschies et al., 2013, Sci Rep 3: 1658). In contrast, the hUC-MSCs treated with 0.05% trypsin showed single cell shape with no connected tissue (FIGS. 11B-D). In addition, the cell surface in 0.05% trypsin treated groups (5 minutes, 20 minutes, and 60 minutes) lost their microvilli-like structure by trypsin treatment-time dependently (FIGS. 11B-D). In the result, hUC-MSC sheet had maintained tissue-like connected structures as well as microvilli-like structures, while proteins on cell surface in 0.05% trypsin treated group were cleaved.

In TEM analysis, hUC-MSC sheet maintained ECMs (white dotted line) and cell-cell junctions (white solid arrow), which are related to cell adhesion and cell-cell communication (Gattazzo et al., 2014, Biochim Biophys Acta 1840(8): 2506-19). (FIG. 11E). However, hUC-MSCs treated with 0.05% trypsin for 5 minutes showed cleaved cell-cell junctions and ECMs, compared to cell sheet group (FIG. 11F). Furthermore, when the hUC-MSCs were treated with 0.05% trypsin for 20 and 60 minutes, hUC-MSCs lost its filopodia on cell surface and had unclear shape of nucleus (FIGS. 11 G and H). The hUC-MSCs treated with 0.05% trypsin for 60 minutes showed endoplasmic reticulum (dark grey arrows) which is known to associate with cell death (FIG. 11 H). SEM and TEM results indicate that hUC-MSC sheet had maintained cell surface proteins and intercellular proteins such as microvilli-like structure, filopodia, ECM, and cell-cell junctions even after cells were detached from cell culture dish. In contrast, hUC-MSCs treated with 0.05% trypsin groups showed cleaved microvilli, ECM, and cell-cell junctions and damaged nucleus. The findings suggest that trypsin treatment (chemical disruption) causes damage to cell and tissue structures (i.e. junction proteins, ECMs, nucleus, and endoplasmic reticulum).

### hUC-MSC Maintains Actin Filament Proteins Relating With Cell Dynamics

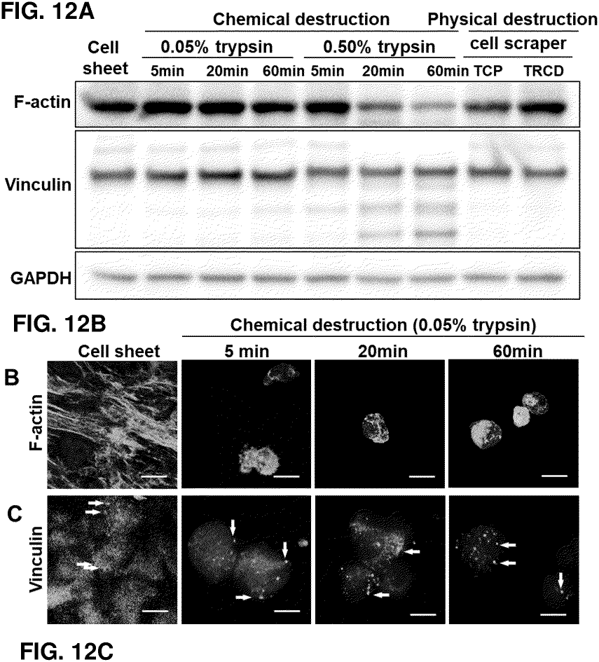

Glyceraldehyde 3-phosphate dehydrogenase (GAPDH) protein expression was detected as a loading control to normalize protein amounts for western blotting assay. GAPDH protein expression level was similar in all groups. Cells treated with 0.50% trypsin for 20 and 60 minutes expressed lower actin than that in cell sheet, 0.05% trypsin, and cell scraper groups (FIG. 12A). This indicates 0.50% trypsin treatment disrupts actin in cytoplasm. To observe cytoskeleton structure, hUC-MSCs were stained with actin. When cells are attached to cultureware surfaces, actin forms stress fiber structures which play an important role in cell survival (Bachir et al., 2017, Cold Spring Harb Perspect Biol 9(7)). Cell sheet groups showed actin stress fiber structure even after cell sheets detached from cell culture dishes. In contrast, 0.05% trypsin treated groups for 5, 20 and 60 minutes showed actin positive area, however stress fiber structures were not observed (FIG. 12B). The amount of F-actin protein was similar in cell sheet and 0.05% trypsin treated groups. However, only cell sheet group had maintained stress fiber structure of actin.

Vinculin is a membrane cytoskeletal protein that forms focal adhesions by linking integrin receptor families and actin, associated with cell movement (Peng, 2011, Int Rev Cell Mol Biol 287: 191-231). Vinculin expression was observed in both cell sheet and 0.05% trypsin treated groups when stained with immunohistochemistry (FIG. 12C). Multiple lower molecular weight bands in western blot analysis of vinculin expression were observed in chemical disruption group (FIG. 12A). This indicates that vinculin proteins were cleaved in the chemical disruption group. The cells treated with trypsin (chemical disruption) revealed delocalized actin fiber structures, reduced actin protein, and cleaved vinculin protein, suggesting chemical disruption method cleaved proteins related to cell shape and cell dynamics. This cleavage was increased when trypsin concentration was increased.

### hUC-MSC Sheet Maintains Extracellular Proteins Related With Cell Adhesion

Fibronectin and laminin are important proteins in cell- and tissue- adhesion. Cell sheet, 0.05% or 0.50% trypsin treatment for 5 minutes, and cell scraper groups in western blot assays expressed fibronectin. However, 0.05% and 0.50% trypsin treatment for 20 minutes, and 60 minutes groups had no expression of fibronectin. Laminin expression was observed in cell sheet, 0.05% trypsin treatment, 0.50% trypsin treatment for 5 minutes, and cell scraper groups. However, 0.50% trypsin treatment groups for 20 minutes and 60 minutes show no detectable laminin expression.

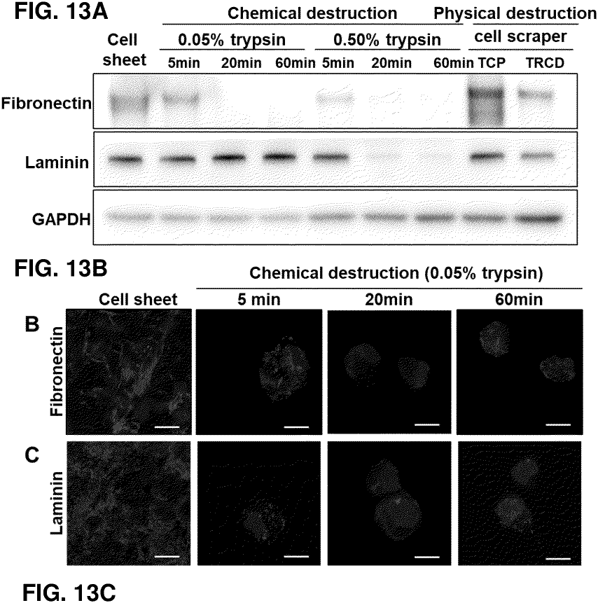

Cells were stained using fibronectin and laminin antibodies to observe the structures of ECM proteins (FIG. 13B). Higher expression of fibronectin was observed in cell sheet group, compared to cells treated with 0.05% trypsin. Cell sheet group showed higher expression of fibronectin and laminin over all cells in cell sheet, similar to tissue structure (fiber structure of ECM). These results suggest that cell sheet group was able to detach cells without disruption of ECM. In contrast, ECM proteins were cleaved with trypsin treatment (chemical disruption) after detachment of cells from cell culture dish.

### hUC-MSC Sheet Maintains Cell Junction Proteins Associated With Cell Communication

Integrin β-1 is the major protein of integrin family which is a membrane protein that forms cell-ECM junctions. It is known that integrins link to actin cytoskeleton through adapter proteins (e.g. vinculin, talin) and are involved in cell survival, cell adhesion and tissue repair (Moreno-Layseca, 2014, Matrix Biol 34: 144-53. Cell sheet, 0.05% trypsin treatment for 5 minutes and cell scraper group showed similar integrin β-1 expression. Integrin β-1 was cleaved gradually as trypsin concentration and treatment time. Connexin 43 is a transmembrane protein that consists gap junction which allows cell-cell communication. Connexin 43 plays essential role in maintaining homeostasis and function of cells and tissues by exchange of biological information (Ribeiro-Rodrigues, 2017, J Cell Sci 130(21): 3619-3630). Connexin 43 was expressed in cell sheet, 0.05% trypsin treated (5, 20, 60 minutes) and 0.5% trypsin treated (5 minutes) groups. However, 0.50% trypsin treatment for 20 and 60 minutes exhibited no expression of connexin 43. This suggests that connexin 43 protein was cleaved by 0.50% trypsin when treated for 20 and 60 minutes.

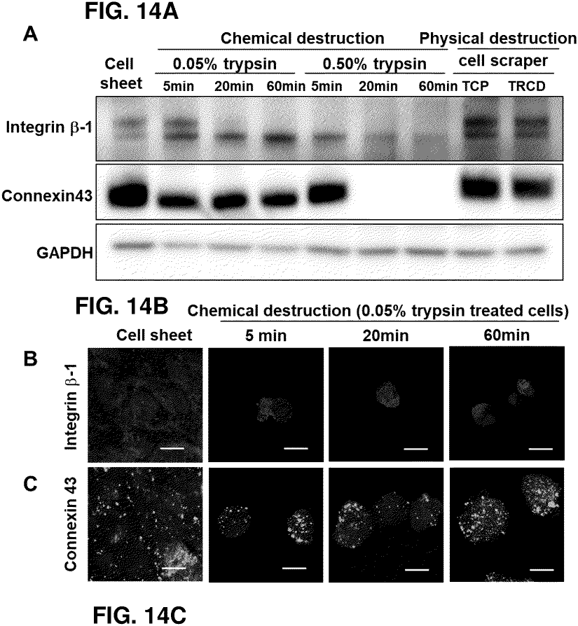

Structural observation of cell junction proteins was performed with integrin β-1 and connexin 43 protein was observed by immunostaining. Cell sheet group showed positive expression of integrin β-1 all over the cell sheet, whereas integrin β-1 was expressed slightly on the cell surface in 0.05% and 0.50% trypsin treated groups (FIG. 14B). Connexin 43 expression was observed in all groups (FIG. 14C). Especially, cell sheet group revealed connexin 43 expression in all over the area. This demonstrates that cell sheet has connected tissue structures and maintain cell-junction proteins. In contrast, trypsin treatment (chemical disruption) cleaved junction proteins.

### Chemical Disruption Method Induces Cell Death

Cells were stained using calcein and ethidium homodimer-1 immediately after cell detachment by trypsin treatment (chemical disruption) or temperature changes (cell sheet technology). Green color shows live cells and red color shows dead cells in FIG. 15. As results, dead cell to live cell ratios in 0.05% trypsin treatment for the 5 and 20 minute groups were similar. Remarkably, dead cell to live cell ratio in the 0.05% trypsin treatment for 60 minutes groups significantly increased, compared to cells treated with 0.05% trypsin for 5 and 20 minutes (FIG. 15B). This result suggests that cell death was induced by trypsin treatment (chemical disruption).

### Apoptotic Cell Death Is Activated by Chemical Disruption

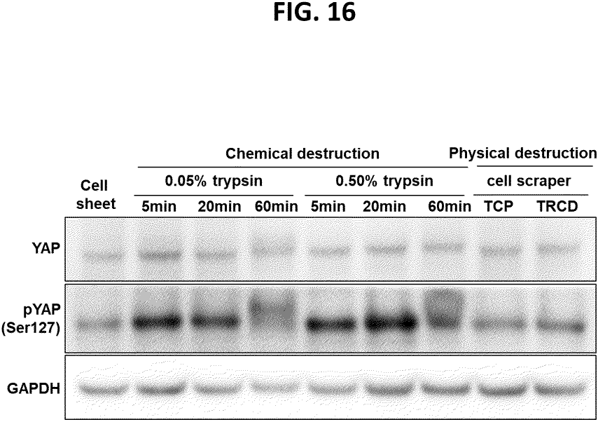

Mechanosensor controls cellular homeostasis by converting extracellular physical stimuli to intracellular chemical stimuli (Humphrey, 2014, Nat Rev Mol Cell Biol 15(12): 802-12). Yes-associated protein (YAP) is one of the major cell mechanosensor proteins and localized at the cell nuclei to regulate cell survival and proliferation (Jaalouk, 2009, Nat Rev Mol Cell Biol 10(1): 63-73). YAP is inhibited via phosphorylation of Ser127 (phosphor-YAP, pYAP), which results in cytoplasmic retention and induction of apoptosis. When cells lose cell-ECM junctions, apoptotic cell death namely anoikis is induced subsequently to YAP phosphorylation (Halder et al. 2012, Nat Rev Mol Cell Biol 13(9): 591-600). YAP and phospho-YAP (pYAP) expression of cell sheet, 0.05% and 0.50% trypsin treatment for 5, 20 and 60 minutes and cell scraper group were determined with western blotting (FIG. 16). All groups showed similar YAP protein expression, whereas expression of pYAP was increased in 0.05% and 0.50% trypsin treated cells compared to cell sheet and cell scraper group (FIG. 16A). This demonstrates that trypsin treatment (chemical disruption) inhibited YAP activity and induced phosphorylation of YAP. In addition, induction of pYAP is known to shift the cell responses to apoptosis.

### Discussion

Chemical disruption method is used to harvest cells from a cell culture dish through disruption of extracellular (Huang et al., 2010, J Biomed Sci 17: 36) and intercellular (Besingi, 2015, Nat Protoc 10(12): 2074-80) proteins which relates to cytoskeleton, cell junction, cell metabolism, and cell growth. Hence, cells harvested by the chemical disruption method (trypsin treated cells) had insufficient ECMs necessary to adhere target tissue, and insufficient cell junctions to maintain their cellular functions through graft-host communication (FIGS. 13 and 14). On the other hand, the hUC-MSC sheet harvested by cell sheet technology using TRCD had maintained tissue-like structures such as smooth surface of connected cells, microvilli, ECM, and cell junction (FIGS. 10, 13, and 14).

The TEM results showed that extracellular protein cleavage was observed in cells treated with 0.05% trypsin for 5 minutes in chemical disruption group. Cytoplasmic cleavage was observed in 20 minutes of 0.05% trypsin treated cells and cell nucleus has degraded at 60 minutes of 0.05% trypsin treatment. In addition, endoplasmic reticulum which relates to cell death was observed at 60 minutes of 0.05% trypsin treatment (FIG. 11). Integrin is the key protein involved in the interaction between cell membrane and ECMs which also links to cytoskeleton (actin) and forms focal adhesion (Kim et al., 2011, J. Endocrinol 209(2): 139-51). Chemical disruption induced cleavage of integrin β-1 as well as cytoskeleton (F-actin), focal adhesion protein (vinculin), ECMs (fibronectin and laminin) (FIGS. 12, 13, and 14). On the other hand, hUC-MSC sheets maintained all integrin β-1, cytoskeleton, focal adhesion protein (vinculin) and ECMs (fibronectin and laminin) even after detachment from cell culture dishes (FIGS. 12, 13, and 14). These findings suggested that chemical disruption method (trypsin treatment) may cause a harsh environment for cell survival due to proteolytic disruption of cell-ECM junction by enzyme.

Yes-associated protein (YAP) has an important role in regulating cell adhesion, proliferation and survival. It is known that apoptotic cell death is induced through inhibition of YAP and subsequent pYAP induction. Similarly, breakdown of cell-ECM junction induces apoptotic cell death through inhibition of YAP (Codelia, 2012, Cell 150(4): 669-70). When cells were treated with trypsin (chemical disruption), integrin β-1 was cleaved (FIG. 14) and the cleavage of integrin β-1 inactivated YAP and induced pYAP (FIG. 16). Eventually, apoptotic cell death occurs in the chemical disruption group (FIGS. 11, 15, and 16). In contrast, the hUC-MSC sheet maintains integrin β-1 and lower expression of pYAP (FIGS. 14 and 16) show significantly higher cell survival rates (FIGS. 15 and 16). It is reported that pYAP can be induced by not only integrin β-1 cleavage but also inhibition of F-actin polymerization [52, 53]. The hUC-MSC sheet showed cytoskeleton fiber structures of F-actin indicating active actin polymerization even after cell detachment from cell culture dish (FIG. 12). This suggests that hUC-MSC sheet retains integrin β-1 (cell-ECM junction) and F-actin structures, which enabled cell sheets to maintain higher cell survival rates compared to trypsin treatment (conventional chemical disruption method). These findings suggest that cell-ECM junction and actin fiber structures are important factors for cell survival and there is difficulty to maintain high cell survival rates using chemical disruption methods.

This study demonstrated that ECM, cell-cell junction and cell-ECM junction proteins are important in retaining higher cell survival rates. In conventional stem cell therapy that uses chemical disruption methods for harvest, it is not possible to avoid low engraftment rate and low cell survival rate, since chemical disruption (e.g. trypsin treatment) cleaves cell-cell junctions, cell-ECM junctions and cell adhesion proteins. Cell sheet technology enabled cells to be harvested as a sheet form without any structural disruption. Furthermore, cell sheet technology maintained important structures of cells (ECMs, cell-ECM junction, cell-cell junction, cytoskeleton and mechanosensors) which relates to cell survival rate, engraftment rate and various cellular functions. As a result, cell survival rate in hUC-MSC sheet was significantly higher than that in the cells harvested with chemical disruption method.

### Conclusion

We demonstrated that tissue-like structure such as ECMs cell-cell junction and cell-ECM junction are associated with cell survival rates of transplanted cells. Cell sheet technology allows culture and harvest of cells as a sheet form without using any enzymes (chemical disruption). The harvested mono-layer hUC-MSC sheet retaining tissue-like structures, ECMs, cell-cell junctions and cell-ECM junctions had higher cell survival rates, compared to conventional chemical disruption method (trypsin treatment). This technology will provide not only higher therapeutic effect of stem cell therapy, but also new concept of functional cells in regenerative medicine research since cell sheet mimics native tissue-like structure.

### Example 3. Gene Expression in Human Umbilical Cord Mesenchymal Stem Cell (hUC-MSC) Sheets

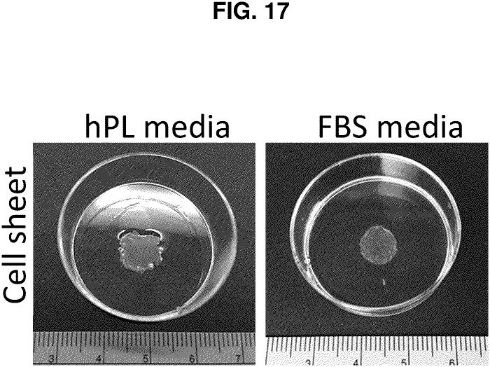

Cell sheets were prepared from hUC-MSCs by the methods described in Example 1 above, except that the cell culture medium contained either 20% hPL or 20% FBS. The hUC-MSC sheets are shown in FIG. 17. Single cell suspension cultures of hUC-MSCs were prepared by culturing hUC-MSCs on cell culture dishes and treating the cells with trypsin (TryLE, Gibco) when they were confluent. The trypsinized single cell suspension of hUC-MSCs was analyzed by flow cytometry.

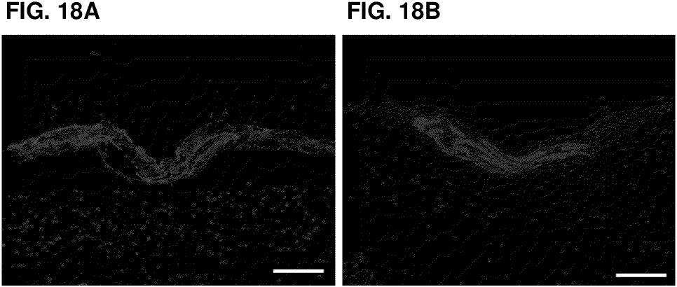

hUC-MSC sheets were cultured in medium containing 20% hPL and implanted within the subcutaneous tissue of immuno-deficient mice as described in Example 1 above, and the hUC-MSC sheets were harvested from the subcutaneous tissue sites for histological observation at 1 day and 10 days after implantation. After harvest, the samples were stained with human growth factor (HGF) antibody for detection of HGF expression, and cell nuclei were stained with DAPI. As shown in FIG. 18, the hUC-MSC sheets expressed HGF 1 day after implantation, and still maintained significant HGF expression 10 days after implantation. These results suggest that hUC-MSC sheets maintain continuous expression of HGF for at least 10 days after implantation into the tissue of a host organism.

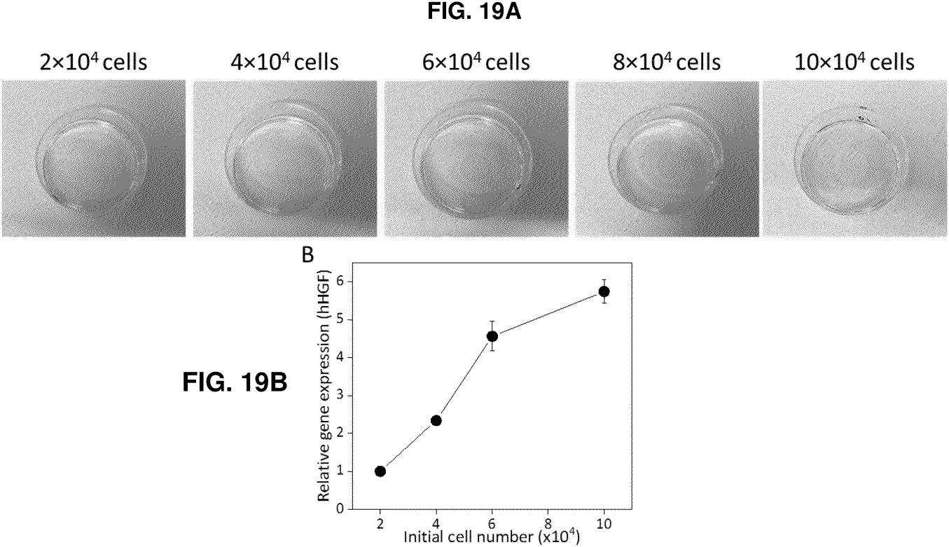

The effect of initial cell density on HGF expression in hUC-MSC sheets was also determined. Cell sheets were prepared from hUC-MSCs in TRCD with an initial cell density of 2×10⁴, 4×10⁴, 6×10⁴, 8×10⁴ or 10×10⁴ cells/cm² in cell culture medium containing 20% FBS. As shown in FIG. 19, increasing the initial cell density increased HGF expression in a dose dependent manner. For example, the cell sheets produced with 10×10⁴ cells/cm² had higher HGF gene expression, compared to the cell sheets produced with 2×10⁴, 4×10⁴, 6×10⁴, 8×10⁴ or 10×10⁴ cells/cm². These results suggest that HGF expression levels in the hUC-MSC sheet can be optimized by controlling the initial cell density in the cell culture support (e.g. the TRCD).

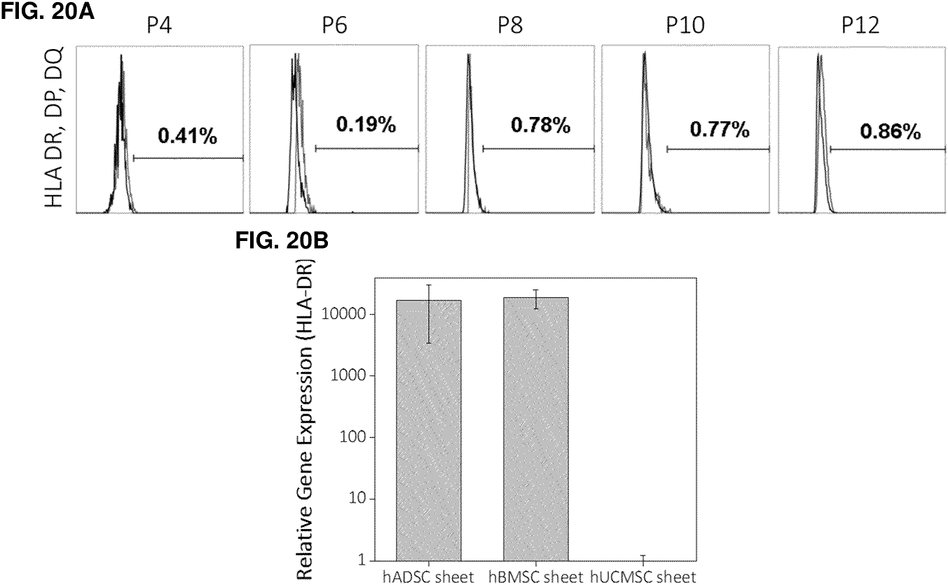

HLA DR, DP, DQ expression was determined in hUC-MSCs in suspension cultures from passage 4 to 12, and in cell sheets prepared from human adipose-derived mesenchymal stem cells (hADSC), human bone marrow-derived mesenchymal stem cells (hBMSC), or hUC-MSCs. Cells were grown in culture medium containing 20% hPL. HLA expression was determined as described above in Example 1. As shown in FIG. 20A, hUC-MSCs maintained low HLA DR, DP, DQ cell surface expression from passage 4 to 12 in cell suspension cultures. As shown in FIG. 20B, HLA-DR gene expression was not detectable in hUC-MSC sheets, while cell sheets prepared from hADSC or hBMSC exhibited relatively high levels of HLA-DR gene expression. Low HLA expression is desirable for reducing an immune response to cell sheets transplanted to a host organism. Accordingly, these results suggest that hUC-MSC sheets are less likely to induce an immune response in a host organism after transplantation relative to cell sheets produced from hADSCs or hBMSCs.

### Example 4. Media Effects on Allogeneic Human Umbilical Cord Mesenchymal Stem Cell (hUC-MSC) Sheet Fabrication

In this study, we compared fetal bovine serum (FBS) and human platelet lysate (hPL), the most common media used for MSC culture, on MSC differentiation ability, MSC-specific phenotypes, cell sheet preparation, cell sheet specific traits, and paracrine secretion.

### Materials and Methods

### Human Umbilical Cord Mesenchymal Stem Cell (hUC-MSC) Culture

Banked hUC-MSCs (Jadi Cell LLC, Miami, USA) (Patel et al., 2013, Cell Transplant 22:513-519) were cultured in cell culture media with Dulbecco’s Modified Eagle’s Medium (DMEM) (Life Technologies, USA) supplemented with either 10% hPL (Jadi Cell LLC) or 10% FBS (Life Technologies), 1% Glutamax (Life Technologies), 1% MEM NEAA (Life Technologies), 1% penicillin streptomycin (Life Technologies), at 37° C. in a humidified atmosphere with 5% CO₂ from passage 2 (P2). The working cell bank was established at P4. Cell culture media was changed every two days. Cell morphologies were observed at P6 and P12 using an AX10 microscope (Carl Zeiss Microimaging, Germany) with AxioVision software (Carl Zeiss Microimaging) before thermally induced cell sheet detachment from cultureware.

### hUC-MSC Proliferation Rates

Cells were seeded at 2.3×10⁴/cm² cell density on 6-well plates. Cell numbers for both FBS or hPL cultures were counted at 8 h, 24 h, 33 h, 48 h, and 72 h after seeding at P4 - 12. Cell doubling time was calculated by polynomial fitting using Origin Pro 2017 (OriginLab, Massachusetts, USA).

### MSC Differentiation Ability

Cells were seeded on 6-well plates for assessing osteogenic or adipogenic differentiation in passage 6. For osteogenic differentiation, cells were plated at 5×10³ cells/cm². When 60% confluent, cells were induced with osteogenic differentiation media containing αMEM, 10 nM dexamethasone, 82 µg/mL ascorbic acid 2-phosphate, 10 mM β-glycerolphosphate (Sigma-Aldrich). Cells were cultured in osteogenic media at 37° C. for 21 days with media changed every 3 days. Cells were fixed with cold 4% paraformaldehyde (PFA) for 12 minutes and stained with Alizarin Red S- (Sigma-Aldrich) using standard protocols. For adipogenic differentiation, cells were plated at 1×10⁴ cells/cm². When 80% confluent, cells were induced with adipogenic differentiation media containing high-glucose DMEM, 100 nM dexamethasone, 0.5 mM IBMX, and 50 µM IND (all Sigma-Aldrich). Cells were cultured in adipogenic media at 37° C. for 21 days and media changed every 3 days. The cell were fixed with cold 4% paraformaldehyde for 12 minutes and stained with Oil Red O (Sigma-Aldrich) using standard protocols.

### Stem Cell Surface Phenotyping Assay

hUC-MSCs were cultured in cell growth media on tissue culture flasks (Genesee Scientific, CA, USA) for 5 days and then detached using TrypLE (Gibco, Waltham, MA, USA) for MSC phenotyping assay. Post-harvest, cell suspensions were incubated with 2% w/v bovine serum albumin (BSA) in PBS for 30 min, then aliquoted at concentrations of 3 - 5×10⁵/100 µL. One aliquot was reserved as an unstained control and those remaining were stained with the following antibodies: CD73, CD105, CD90, MHC II, CD45, and CD31 (Biolegend, San Diego, USA). Primary antibody was added to each aliquot to achieve a ratio of about 20:1 of cells in buffer to antibody. About 3 - 5×10⁵ cells were stained with saturating concentrations of (fluorophore)-conjugated primary antibodies. Cells were incubated in the dark on ice for 30 minutes. After incubation, cells were washed three times and then resuspended in 1X PBS and immediately analyzed by flow cytometry (Becton Dickinson FACS Canto, BD Biosciences, Sparks, USA). Flow cytometer calibration used unstained cells. Cells were gated by forward versus side scatter to eliminate doublets. A minimum of 10,000 events was counted for each analysis.

### hUC-MSC Sheet Preparation

hUC-MSC sheets were prepared using P6 cells. These cells were seeded at 2.3×10⁴/cm² density on 35-mm temperature responsive cell culture dishes (TRCD, CellSeed Inc., Tokyo, Japan) and cultured in cell culture media with DMEM supplemented with either 20% FBS or 20% hPL at 37° C. in a humidified atmosphere with 5% CO₂ for 4 (FBS) or 3 (hPL) days, respectively. Ascorbic acid (16.4 µg/ml) was added one day after seeding. Confluent cells were detached by changing culture temperature from 37° C. to room temperature (RT) within 1 h at 3 or 4 days after seeding to analyze cell sheet properties. Sheet spontaneous surface detachment time was determined when cell TRCDs were moved to RT from 37° C. incubation without any additional manipulations, such as pipetting, scraping or media changes.

### Histological Analysis

For H&E staining, cell sheets were fixed with 4% PFA for 15 min and then embedded in paraffin. Embedded specimens were sectioned into 4 µm slices. Specimens were treated with hematoxylin solution (Sigma-Aldrich) for 3 min and subsequently with eosin solution (ThermoFisher Scientific, Waltham, USA) for 5 min. H&E stained specimens were dehydrated and mounted with Permount™ (ThermoFisher Scientific). H&E images were obtained using a BX 41 microscope (Olympus, Hamburg, Germany). For immunohistochemistry (IHC), cells were fixed when they reached confluence using 4% PFA for 10 min. Cell membranes were permeabilized with 0.1% Triton X (Sigma-Aldrich, St. Louis, USA) for 15 min. Non-specific binding was blocked in PBS 1X containing 10% goat serum (Vector Laboratories, Burlingame, USA) for 1 h at room temperature. Cells were stained with Alexa Fluor 488® phalloidin (ThermoFisher Scientific) to visualize cytoskeleton, or with primary antibodies (Abcam, Cambridge, USA) to image fibronectin (Fb, ab2413) (Abcam, Cambridge, USA), β-catenin (β-CTNN, ab16051) (Abcam), HLA DR, DP, DQ (MHC II, ab7856) (Abcam), and negative control rabbit IgG (NC, x0936) (DAKO, Santa Clara, CA). These specimens were treated with Alexa Fluor 594-conjugated secondary antibodies (Life Technologies) (1:200) for 1 h for Fb, β-CTNN, and MHC II stains. Stained cells were mounted with ProLong Gold Antifade Reagent including DAPI (Life Technologies). Immunofluorescence images were obtained using an AX 10 microscope (Carl Zeiss Microimaging) and analyzed with Axiovision software (Carl Zeiss Microimaging).

### Gene Expression Analysis of Cell Sheets

Total RNA from cell sheets was extracted using Trizol and PureLink RNA Mini Kit (Life Technologies, Carlsbad, USA) according to manufacturer’s protocols. cDNA was prepared from 1 µg of total RNA using high capacity cDNA reverse transcription kits (Life Technologies). qPCR analysis was performed with TapMan Universal PCR Master Mix using an Applied Biosystems Step One instrument (Applied Biosystems™, Foster City, USA). Gene expression levels were assessed for the following genes: 1) glyceraldehyde 3-phosphate dehydrogenase (GAPDH, Hs99999905_ml) as a housekeeping gene, 2) β-actin (Hs999999903_ml), 3) integrin β-1 (ITGB1, Hs01127536_ml), 4) fibronectin (Fb, Hs01549976_ml), 5) β-catenin (Hs00355049_ml), and 5) major histocompatibility complex II (MHC II: HLA-DRB, Hs04192464_ml). All primers were manufactured by Applied Biosystems. Relative gene expression levels were quantified by the comparative CT method (Schmittgen et al., 2009, Nat Protoc 3:1101-1108). Gene expression levels were normalized to GAPDH expression levels. Gene expression levels are relative to the hPL group for β-actin and ITGB1 analysis and to the FBS group for Fb, β-CTNN, and MHC-II analysis.

### Hepatocyte Growth Factor (HGF) Secretion Assay

Supernatant media of adherent cells cultured for 24 hours was collected just prior to cell sheet detachment from TRCDs at RT. HGF amounts secreted from cell sheets were measured by human HGF Quantikine ELISA (R&D Systems, Minneapolis, USA) according to manufacturer’s protocols. Cells were detached with TrypLE to count cell numbers comprising each cell sheet. HGF amount was normalized to cell sheet or cell numbers.

### Statistical Analyses

All quantitative values are expressed as mean and standard error (SE, mean±SE). Significant differences between groups were tested by one-way Analysis of Variance ANOVA) using Origin 2017 software (OriginLab, Northampton, USA). A probability value of less than 0.05 (p < 0.05) was considered statistically significant.

### Results

### Changes in hUC-MSC Morphology and Growth Rate

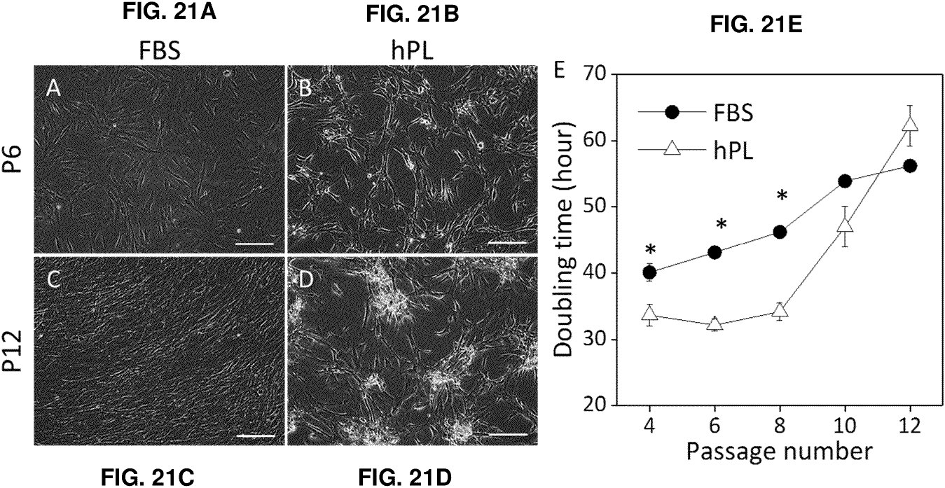

MSCs were cultured in cell culture media including FBS or hPL and observed at 1 day and 4 days after seeding in P6 (FIGS. 21A and 21B) and P12 (FIGS. 21C and 21D), respectively. MSCs in hPL media showed more elongated spindle shapes, while MSCs in FBS were flatter. (FIGS. 21A and 21B). hUC-MSCs cultured in FBS media grew in homogeneous dispersions, while those in hPL media tended to grow in clumped dispersions (FIGS. 21A-21D). Clumped dispersive growth of hPL groups is more evident as passage number increased, especially for P12 (FIG. 21D). Cells in the hPL groups showed significantly higher proliferative ability compared to those in FBS groups from P4 to P8. However, the doubling times of cells in hPL groups rapidly increases after P8 (FIG. 21E).

### Differentiation Potential and MSC Cell-Specific Phenotypes

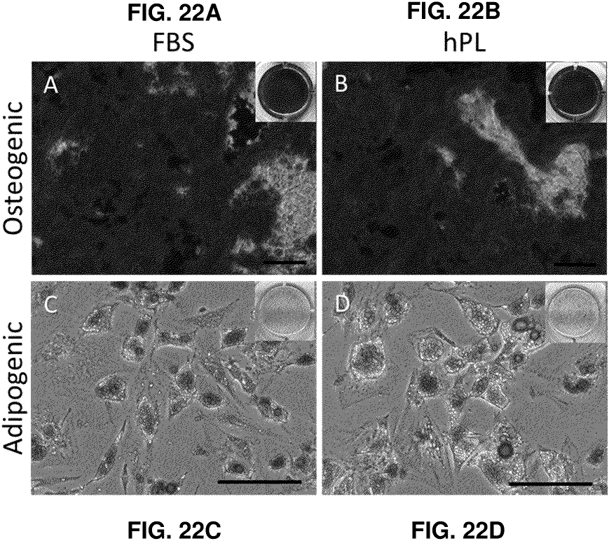

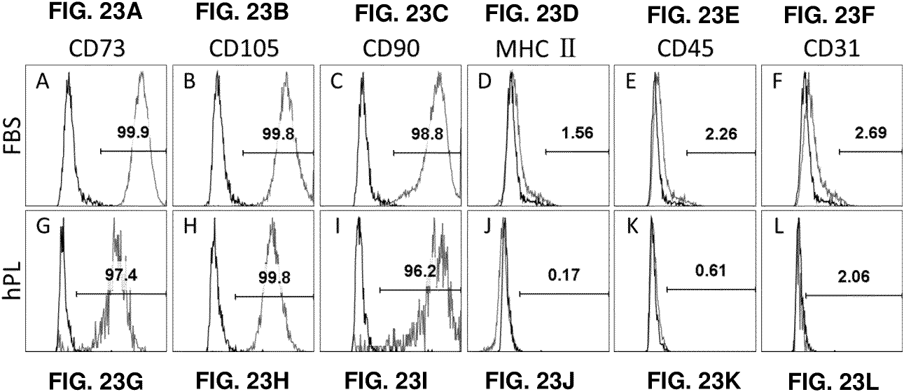

To assess how FBS and hPL media affects hUC-MSC differentiation potential and stem cell-specific phenotypes, differentiation potential and stem cell surface markers were assessed after culture for two passages in FBS (FIGS. 22A and 22C) or hPL (FIGS. 22B and 22D) media. Differentiation potential in both osteogenesis and adipogenesis was not affected by FBS or hPL media (FIGS. 2A-2D). Also, cells cultured in both FBS (FIGS. 23A-23F) or hPL (FIGS. 23G-23L) showed stem cell-specific surface markers; positive expression for CD73, CD105, CD90 (FIGS. 3A-3C and 3G-3I) and negative expression for MHC II, CD45, CD31 (FIGS. 23D-23F and 23J-23L).

### Cell Sheet Fabrication

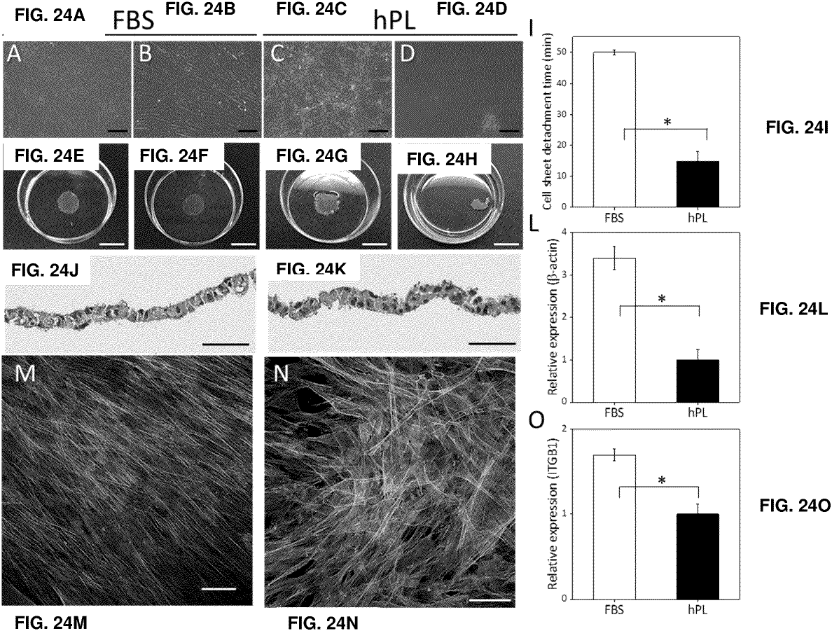

Cell sheets were prepared in cell culture media including 20% FBS or hPL. Cells in FBS or hPL groups reached confluence on TRCD at 4 or 3 days, respectively, after seeding and then were harvested as sheets detached using small temperature changes from 37° C. to RT (FIGS. 24A-24H) without additional manipulations. Cell sheets cultured in FBS media were recovered when cells were over-confluent (FIGS. 24B and 24F), while cell sheets cultured in hPL media detached spontaneously at 37° C. more as cell clumps (FIGS. 24D and 24H). Cell sheets in FBS and hPL groups were recovered within 50 min and 15 min at RT, respectively (FIG. 24I). Cells in hPL groups exhibited denser cell nuclei in cell sheets compared to those in FBS groups (FIGS. 24J and 24K). Actin cytoskeleton (F-actin) for cells in FBS groups was aligned and well-organized (FIG. 24M). On the other hand, that in hPL group exhibits a criss-cross random actin cytoskeleton pattern (FIG. 24N). Gene expression levels for β-actin and ITGB1 related to stable cell adhesion in FBS groups were significantly higher than in hPL groups (FIGS. 24L and 24O). Data indicate that hPL cell sheets grow rapidly to high density, exhibit faster detachment at reduced temperature, weaker cell adhesion, and tend to form cell aggregates even without temperature reduction more readily upon release under poorly controlled sheet production conditions.

### Phenotypic Traits of hUC-MSC Cell Sheets

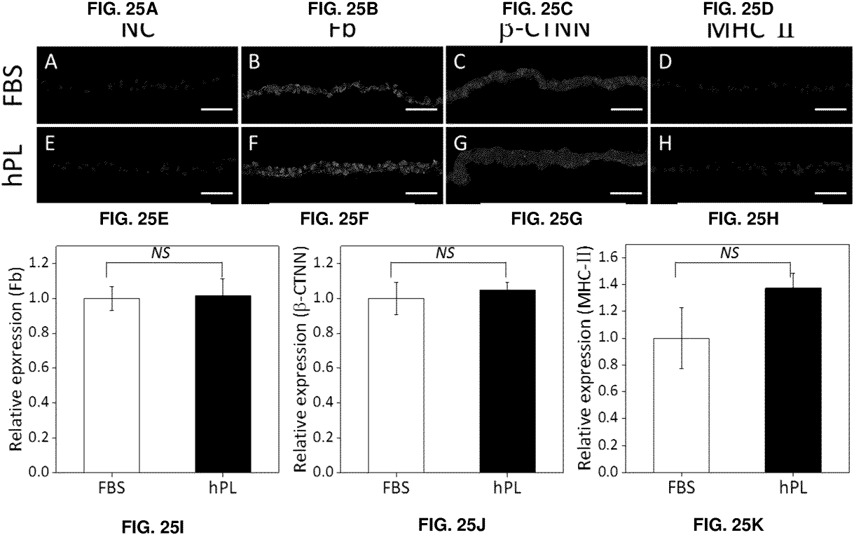

Desired cell sheet-specific traits (e.g., retention of endogenous cell adhesive proteins), correlating to therapeutic and engraftment efficacy, for cell sheets prepared with FBS and hPL media were assessed by IHC analysis. Both cell sheets in FBS and hPL groups showed positive expression of cell adhesive proteins (ECM: Fibronectin (Fb) and cell junction protein: β-catenin (β-CTNN)), compared to negative control (NC) (FIGS. 25A-25C and 25E-25G). Also, gene expression levels for Fb and β-CTNN were assessed thorough q-PCR analysis and were all similar in both FBS and hPL groups (FIGS. 25I and 25J).

### Histocompatibility Markers for hUC-MSC Sheets

MHC II expression is an indicator of possible host immune reactivity when allogeneic cells are implanted in vivo (Zantvoort et al., 1996, Transplantation 61:841-844.). MHC II positive expression is barely detectable for cell sheets prepared in FBS and hPL groups (FIGS. 25D and 25H). Gene expression levels related to MHC II were also similar in both FBS and hPL groups (FIG. 25K). These data demonstrate that neither FBS nor hPL media culture conditions activate this immune response antigen.

### Cell Sheet Paracrine Factor Secretion

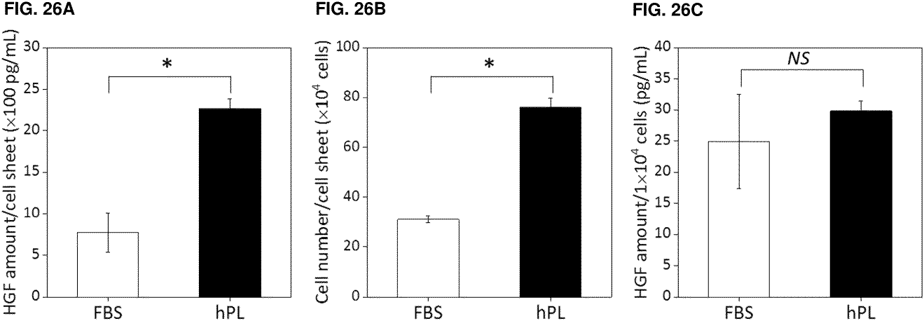

Human growth factor (HGF) secreted from each cell sheet condition for 24 hours was measured in media supernatant. hPL cell sheets secrete significantly more HGF compared to FBS cell sheets (FIG. 26A). When secreted HGF is normalized to cell number (FIG. 26B) per cell sheet, HGF amounts in FBS and hPL groups are similar because hPL cell sheets comprise more cells than FBS cell sheets (FIG. 26C). This result indicates that increased paracrine factor secretion from hPL versus FBS cell sheets is attributed to their higher proliferation ability (FIG. 26B) and resulting higher cell density per sheet (FIG. 26C).

### Discussion

Many MSC studies, including their current diverse clinical applications, use media supplemented with FBS to promote cell attachment, growth, and provide vital nutrients during MSC culture and storage. To date, FBS culture is accepted as clinical-scale cell production method. However, xeno-derived media and additives carry known risks of contamination from prions, viral, and zoonotic agents as well as possible host immune reaction stimulus. Recently, hPL has been considered as an alternative to FBS for avoiding potential immune cross-reactions. Also, some data indicate that hPL supports greater MSC proliferative abilities than FBS for large-scale MSC production. In this present study, MSC culture in hPL media exhibited greater proliferation rates (1.2-1.4 times higher) for early passage numbers (i.e., between P4 and P8) than MSC cultured in FBS media (FIG. 21E). However, the MSC proliferation rate after P8 in hPL was similar to that in FBS media. hPL media induces wide differences in MSC proliferation rates for different passage numbers (FIG. 21E). Additionally, both FBS and hPL conditions maintain cell sheet differentiation potential and phenotypic traits characteristic of MSCs (FIGS. 22 and 23). Thus, cell culture media including hPL that prompt higher cell proliferative capacities without altering MSC-specific traits in early passage numbers would be valuable for large-scale MSC production. However, cell sheets in hPL media exhibit unexpected detachment from TRCD without any temperature changes when cells become over-confluent, possibly induced by higher cell density within the cell sheet (FIG. 24H). Therefore, cell sheet fabrication protocols using hPL media should be standardized with specific attention to culture time to avoid unexpected cell sheet detachment during sheet fabrication.

In this study, even though the precise mechanism is unclear, MSCs in hPL media exhibit randomly arranged multi-directional actin cytoskeleton pattern while MSC cytoskeletal features in FBS media are aligned unidirectionally (FIGS. 24M and 24N). Criss-cross, random actin cytoskeletal structure causes more contractile forces because contraction of cells with randomly aligned cytoskeletal structures occur from multiple directions simultaneously, while cells with unidirectionally aligned cytoskeletal structures contract only from one direction (Takahashi et al., 2015, Adv Healthc Mater 4: 2388-240). Cytoskeletal structure is also well-recognized to contribute to cell morphologies. MSCs in hPL show thinly elongated cell shapes with criss-cross, randomly arranged actin cytoskeletal structures. MSCs in FBS media cover larger areas with flattened cell shapes (FIGS. 21A-21B and 24M-24N). Furthermore, cytoskeletal structures were correlated to cell adhesion because ITGB1 binding to the actin cytoskeleton is involved in cell adhesion (Fernandez-Rebollo et al., 2017, Sci Rep 7:5132). In the study, MSCs in FBS media expressed more actin and ITGB1 markers compared to MSCs in hPL media (FIGS. 24L and 24O). Some studies also demonstrate that MSCs in hPL media produce more spindle-shaped fibroblast-like morphologies and weaker cell adhesion to tissue culture plastics compared to MSCs in FBS media (Fernandez-Rebollo et al., 2017, Sci Rep 7:5132). These characteristics suggest that their aligned cytoskeletal structures in FBS media provide stable cell morphologies and adhesion. Cell sheet fabrication produces confluent cells adherent under cell-cell tension on TRCDs and harvested as a contiguous sheet from these rigid culture surfaces at RT. Spontaneous thermally induced cell sheet detachment from this tensile, adherent state produces spontaneous cell sheet contraction through endogenous cytoskeletal contraction upon release from the TRCD. MSC sheets derived from hPL media detach rapidly from the TRCD within 15 min; faster than cell sheets in FBS media (detachment time: 49 min) (FIG. 24I). These differences in MSC sheet detachment time from TRCD in FBS versus hPL media likely result from combinations of weaker MSC adhesion to TRCD surfaces and rapid contraction within MSC sheets due to criss-cross random cytoskeletal structures in hPL cultures. Overall, hPL media rapidly produces MSC sheets through random actin cytoskeleton and weaker cell adhesion, but with unexpected strong cell-cell contractile responses after non-temperature dependent cell sheet detachment.

Recently, cell-based therapies increasingly focus on allogeneic stem cell sources because of several advantages: 1) reducing high production costs for autologous sourcing, 2) reducing donor variability (e.g., aging and disease pathophysiology) affecting stem cell quality, and 3) allowing cell-banked sourcing of healthy donors for “off-the-shelf” products. However, allogeneic cell therapies are also currently limited by host-patient immunologic competence and transplant compatibility (as assessed through major histocompatibility complex, MHC), and inconsistent cell delivery methods to effectively produce both cell retention and therapeutic engraftment. This study uses hUC-MSCs shown to present low antigenicity/histocompatibility profiles (MHC) (Patel et al., 2013, Cell Transplant 22:513-519). Consistent with this, MSC sheets prepared in both FBS and hPL media expressed low MHC class II antigens (FIG. 23), and maintained low MHC class II antigen expression during cell sheet preparation (FIGS. 25D and 25H). Cell sheets prepared with FBS and hPL also retain critical cell adhesive proteins related to therapeutic and engraftment efficacy (FIGS. 25D, 25C, 25F and 25G). No significant differences in gene expression levels for critical cell adhesive protein production are seen between FBS and hPL groups (FIGS. 25I and 25J). Overall, FBS and hPL media retain MSC phenotypic traits and structural features needed for cell sheets deemed important to host immune histocompatibility and optimal therapeutic and engraftment processes. These findings suggest that cell culture media supplemented with either FBS and hPL could be used in standard production methods to maintain cell sheet-specific traits.

HGF is a cytokine important to mechanisms that inhibit fibrosis and promote tissue repair in vivo (Inoue et al., 2003, FASEB J 17:268-270). At similar MSC seeding densities (2.3×10⁴ cells/well), MSC sheets prepared with hPL media secrete higher HGF levels than those prepared with FBS media (FIG. 26A). On TRCD, hPL groups produce 2.5 times higher final cell numbers in MSC sheets compared to FBS groups (FIG. 26B). HGF amounts normalized to final cell sheet numbers for each media were similar in both FBS and hPL groups (FIG. 26C), indicating that increased HGF secretion from hPL-produced cell sheets is due to higher cell proliferative capacities and resulting cell sheet number differences in hPL-cultured sheets. Based on this, it is possible that MSC cell sheet cytokine secretion can be controlled via cell density and proliferative potential in culture. Future studies will investigate influences of MSC cell density in cell sheets on paracrine production and signaling.

We have demonstrated cell culture media influences on certain MSC sheet properties important for eventual cell sheet production, standardization and quality control. MSC-specific biomarkers, secretory products, phenotypic traits and immunogenic antigen/histocompatibility profiles will be essential to determine and establish for these MSC-specific sheet processes. Media supplemented by FBS and hPL both maintain MSC phenotypic traits and cell sheet-specific structures (e.g., cell adhesive proteins) shown to be important for their in vivo therapeutic and engraftment efficacy (Sekine et al., 2011, Tissue Eng Part A 17:2973-2980). However, final cell sheet product consistency prepared in hPL media was lower than that prepared in FBS media: cell sheets cultured in hPL media spontaneously detach from TRCDs without the desired, applied temperature-releasing trigger, and when they release, often yield a MSC clump instead of a stable MSC sheet. This is asserted to be due to weaker cell adhesion and higher contractile forces within the sheets by cytoskeletal strucutre changes. HGF cytokine secretion is increased in MSC sheets prepared in hPL media versus FBS media due to increased final cell sheet numbers in hPL-culture.

### Conclusions

MSCs cultured in FBS and hPL exhibit different cytoskeletal structures. This feature affects cell adhesion to TRCD surfaces and cell sheet contractile forces, important aspects of the cell sheet fabrication process. FBS media MSC culture shows advantages in consistent MSC cell sheet production, with stable cell adhesion to TRCD, phenotypic stability and controlled contractile forces within released harvested MSC sheets. hPL media MSC culture enables more rapid cell sheet production and enhanced HGF cytokine secretion. Both FBS and hPL media retain desirable MSC cell sheet-specific traits essential for their eventual therapeutic goals and efficacy. These findings provide an initial foundation to establish standardized MSC sheet fabrication methods essential for large-scale MSC sheet manufacturing for eventual clinical studies.

We claim:

1. A human umbilical cord mesenchymal stem cell sheet comprising one or more layers of confluent human umbilical cord mesenchymal stem cells (hUC-MSCs).

2. The cell sheet of claim 1, wherein the cell sheet consists essentially of hUC-MSCs.

3-5. (canceled)

6. The cell sheet of claim 1, wherein the cell sheet comprises cell adhesion proteins and cell junction proteins, wherein the cell junction proteins are selected from the group consisting of Vinculin, Integrin-β1, Connexin 43, β-catenin, Integrin-linked kinase and N-cadherin.

7. (canceled)

8. The cell sheet of claim 1, wherein the hUC-MSCs are isolated from the subepithelial layer of human umbilical cord tissue.

9. (canceled)

10. The cell sheet of claim 1, wherein the hUC-MSCs express one or more cytokines selected from the group consisting of human growth factor (HGF), vascular endothelial growth factor (VEGF) and interleukin-10 (IL-10), and wherein expression of the one or more cytokines in the cell sheet is increased relative to a suspension of hUC-MSCs containing an equivalent number of cells.

11-12. (canceled)

13. The cell sheet of claim 10, wherein the cell sheet expresses the one or more cytokines for at least 10 days after transplantation to a tissue in a host organism.

14. The cell sheet of claim 1, wherein the cell sheet expresses extracellular matrix proteins and cell junction proteins for at least 10 days after transplantation to a tissue in a host organism, wherein the extracellular matrix proteins are selected from the group consisting of fibronectin, laminin and collagen, and wherein the cell junction proteins are selected from the group consisting of Vinculin, Integrin-β1, Connexin 43, β-catenin, Integrin-linked kinase and N-cadherin.

15-16. (canceled)

17. The cell sheet of claim 1, wherein initial seeded cell density of the hUC-MSCs in a cell culture support used to prepare the cell sheet is from 0.5 × 10⁴/cm² to 9 × 10⁵ /cm².

18. The cell sheet of claim 1, wherein the hUC-MSCs do not express one or more of CD31, CD45, Human Leukocyte Antigen - DR isotype (HLA-DR), Human Leukocyte Antigen - DP isotype (HLA-DP), or Human Leukocyte Antigen -DQ isotype (HLA-DQ).

19. (canceled)

20. The cell sheet of claim 1, wherein the cell sheet remains attached to a tissue in a host organism for at least 10 days after transplantation to the tissue.

21. A composition comprising the cell sheet of claim 1 and a polymer-coated culture support that is removable from the cell sheet.

22. A method for producing a human umbilical cord mesenchymal stem cell sheet comprising one or more layers of confluent human umbilical cord derived mesenchymal stem cells (hUC-MSCs), the method comprising:
a) culturing hUC-MSCs in culture solution on a temperature-responsive polymer which has been coated onto a substrate surface of a cell culture support, wherein the temperature-responsive polymer has a lower critical solution temperature in water of 0-80° C.;
b) adjusting the temperature of the culture solution to below the lower critical solution temperature, whereby the substrate surface is made hydrophilic and adhesion of the cell sheet to the surface is weakened; and
c) detaching the cell sheet from the culture support.

23. The method of claim 22, further comprising culturing the hUC-MSCs through multiple subcultures prior to the culturing step (a).

24-28. (canceled)

29. The method of claim 22, wherein the adjusting step (b) is performed when the hUC-MSCs are confluent.

30. The method of claim 22, wherein the culturing step (a) comprises adding the hUC-MSCs to the culture solution at an initial cell seeding density from 0.5 × 10⁴/cm² to 9 × 10⁵/cm².

31. The method of claim 22, wherein the hUC-MSCs are cultured in the culture solution on the temperature-responsive polymer for at least 24 hours before the adjusting step (b).

32. A cell sheet produced by the method of claim 22 .

33. A method of transplanting a cell sheet to a subject comprising applying the cell sheet of claim 1 to a tissue of a subject.

34. The method of claim 33, wherein the hUC-MSCs in the cell sheet are allogeneic to the subject.

35. The method of claim 33, wherein the subject is a human.
